(19) United States
(12) Patent Application Publication
(10) Pub. No.: US 2024/0261227 A1
(43) Pub. Date: Aug. 8, 2024

(54) Inhalable Pharmaceutical Formulations

(71) Applicants: Michael OGBURN, Lake Forest, CA (US); Christopher PRICE, Monroe Township, NJ (US)

(72) Inventors: Michael OGBURN, Lake Forest, CA (US); Christopher PRICE, Monroe Township, NJ (US)

(21) Appl. No.: 18/561,351

(22) PCT Filed: Jun. 1, 2022
(86) PCT No.: PCT/US2022/031730

§ 371 Date: Nov. 16, 2023

Related U.S. Application Data
(60) Provisional application No. 63/196,439, filed on Jun. 3, 2021.

(51) Int. Cl. A61K 9/50; A61K 9/00; A61K 9/16; A61K 31/137; A61K 38/28

(52) U.S. Cl. CPC A61K 9/5047; A61K 9/0075; A61K 9/1623; A61K 31/137; A61K 38/28

(57) ABSTRACT

Provided herein are pharmaceutical compositions, kits comprising pharmaceutical compositions, methods of treating disease, and methods of making compositions and kits described herein. The pharmaceutical compositions described herein are powdery pharmaceutical compositions. The powdery pharmaceutical compositions may be administered by an inhaler device described herein. The powdery pharmaceutical compositions can comprise encapsulated epinephrine or encapsulated insulin.

### CROSS REFERENCE TO RELATED APPLICATIONS

This application claims the benefit of U.S. Provisional Application No. 63/196,439, filed Jun. 3, 2021 the disclosure of which is incorporated herein by reference in its entirety.

### INCORPORATION BY REFERENCE

All publications, patents, and patent applications herein are incorporated by reference to the same extent as if each individual publication, patent, or patent application was specifically and individually indicated to be incorporated by reference. In the event of a conflict between a term herein and a term in an incorporated reference, the term herein controls.

### SUMMARY

Disclosed herein are powdery pharmaceutical compositions, comprising: i) particles of a pharmaceutically acceptable excipient; and ii) a plurality of spray dried particles, each particle of the plurality of spray dried particles comprising: a) epinephrine or a pharmaceutically acceptable salt thereof, substantially encapsulated in a coating material; or b) an insulin or a derivative thereof, substantially encapsulated in a coating material. In some aspects, within the plurality of spray dried particles at least a portion of the spray dried particles comprising the epinephrine or the pharmaceutically acceptable salt thereof, or the insulin or the derivative thereof, substantially encapsulated in the coating material, individually have a particle diameter ranging from about 1 micrometer to about 10 micrometers, as measured by a particle analyzer using laser diffraction. In some aspects, the coating material may comprise a hydroxypropyl methylcellulose (HPMC), a hydroxypropyl methylcellulose acetate succinate (HPMCAS), a povidone, a copovidone or any combination thereof. In some aspects, each particle of the plurality of spray dried particles comprises the epinephrine or a pharmaceutically acceptable salt thereof. In some aspects, each particle of the plurality of spray dried particles comprises the insulin or the derivative thereof. In some aspects, the powdery pharmaceutical composition may be for inhaled use or for intranasal use. In some aspects, the powdery pharmaceutical composition may be in unit dose form. In some aspects, at least a portion of the particles of the pharmaceutically acceptable excipient individually may have a particle diameter ranging from about 50 micrometers to about 200 micrometers, as measured by a particle analyzer using laser diffraction. In some aspects, the particles of i) and the plurality of spray dried particles of ii) may be admixed into a substantially homologous mixture. In some aspects a powdery pharmaceutical composition may be contained within a capsule. In some aspects, a capsule may be about one quarter to about one half, by volume, filled with the powdery pharmaceutical composition. In some aspects, a weight-to-weight ratio of: a) the particles of the pharmaceutically acceptable excipient and b) the particles comprising the epinephrine or the pharmaceutically acceptable salt thereof, substantially encapsulated in a coating material, may range from about 1:1 (w/w) to about 10000:1 (w/w). In some aspects, a weight-to-weight ratio of: a) the particles of the pharmaceutically acceptable excipient and b) the particles comprising the insulin or the derivative thereof, substantially encapsulated in a coating material, may range from about 1:1 (w/w) to about 10000:1 (w/w). In some aspects, the weight-to-weight ratio of: a) the particles of the pharmaceutically acceptable excipient and b) the particles of the epinephrine or the pharmaceutically acceptable salt thereof, may range from about 1:1 (w/w) to about 10:1 (w/w). In some aspects, the portion of a capsule not containing a powdery pharmaceutical composition may comprise a gas that at least partially comprises an inert gas. In some aspects, an inert gas may comprise nitrogen, carbon dioxide, helium, or any combination thereof. In some aspects, an inert gas may comprise at least about: 80%, 85%, 90%, or 95% of the gas on a volume-to-volume basis. In some aspects, i) the powdery pharmaceutical composition within the capsule, ii) the gas within the capsule, or iii) any combination thereof may comprise less than about 10% water by weight based on the weight of the powdery pharmaceutical composition or a total content of all gases in the capsule may be less than about 10% water by weight within: the powdery pharmaceutical composition within the capsule, the gas within the capsule, or any combination thereof. In some aspects, the capsule may comprise a hydroxypropylmethylcellulose (HPMC) capsule. In some aspects, a capsule may be size: 000, 00, 0, 1, 2, 3, or 4. In some aspects, the capsule may be size 3. In some aspects, in a human clinical trial, when inhaled into lungs, the powdery pharmaceutical composition may operate mechanistically such that in at least a portion of the humans in the clinical trial, a majority of the particles of the pharmaceutically acceptable excipient deposit onto an oropharynx. In some aspects, the powdery pharmaceutical composition may be contained within an inhaler unit. In some aspects, the capsule may be contained in an inhaler unit. In some aspects, the pharmaceutically acceptable excipient may comprise a carbohydrate, an alginate, povidone, a carbomer, a flavor, a natural gum, a silicone, an alcohol, a butter, a wax, a fatty acid, a preservative, a pharmaceutically acceptable salt of any of these, or any combination thereof. In some aspects, the pharmaceutically acceptable excipient or pharmaceutically acceptable salt thereof may comprise the carbohydrate or the pharmaceutically acceptable salt thereof. In some aspects, the carbohydrate or the pharmaceutically acceptable salt thereof may comprise lactose, microcrystalline cellulose, cellulose, mannitol, sorbitol, starch, starch glycolate, hydroxypropyl methylcellulose, hydroxypropyl methylcellulose acetate succinate, a cyclodextrin, maltodextrin, croscarmellose sodium, corn starch, carrageenan, sorbitol, maltitol, glucose, a pharmaceutically acceptable salt of any of these, or any combination thereof. In some aspects, a pharmaceutically acceptable excipient or the pharmaceutically acceptable salt thereof may comprise lactose or a pharmaceutically acceptable salt thereof. In some aspects, the lactose or the pharmaceutically acceptable salt thereof, may comprise milled lactose, sieved lactose, micronized lactose, spray dried lactose, at least substantially anhydrous lactose, monohydrate lactose, a pharmaceutically acceptable salt thereof, or any combination thereof. In some aspects, a powdery pharmaceutical composition described herein when stored in a sealed container placed in a room at 25° C. and a room atmosphere having about 50 percent relative humidity, may retain at least about: 90% of the epinephrine or the pharmaceutically acceptable salt thereof, or the insulin or the derivative thereof after 6 months, as measured by HPLC. In some aspects, the epinephrine or the pharmaceutical acceptable salt thereof may be present in an amount ranging from about 0.1 mg to about 0.5 mg. In some aspects, epinephrine or the pharmaceutically acceptable salt thereof may be in the form of a pharmaceutically acceptable salt thereof and may be a hydrochloride salt, a bitartrate salt or a borate salt. In some aspects, epinephrine or the pharmaceutically acceptable salt thereof may comprise at least about 1% by weight of the overall powdery pharmaceutical composition. In some aspects, the insulin or the derivative thereof can be present in an amount ranging from about 10 μg to about 100 μg. In some aspects, the insulin or the derivative can comprise a rapid-acting insulin, a short-acting insulin, an intermediate-acting insulin, a long-acting insulin, an ultra-long acting insulin, or a mixture thereof. In some aspects, the plurality of spray dried particles can comprise a median diameter of about 1 μm to about 5 μm. In some aspects, the plurality of spray dried particles can comprise a median diameter of about 1 μm to about: 6 μm, 7 μm, 8 μm, or 9 μm. In some aspects, a powdery pharmaceutical composition may comprise a tap density of about 0.7 g/cm³. In some aspects, a powdery pharmaceutical composition may comprise a tap density of more than about: 0.4 g/cm³. In some aspects, particles comprising the epinephrine or the pharmaceutically acceptable salt thereof may comprise a fine particle fraction of less than 5.6 m of at least about 45% of an emitted dose upon aerosolization. In some aspects, particles comprising the epinephrine or the pharmaceutically acceptable salt thereof may comprise a fine particle fraction of greater than 6 m of at least about 56% of an emitted dose upon aerosolization.

Also disclosed herein are kits comprising the powdery pharmaceutical composition disclosed herein contained at least in part in a packaging.

Also disclosed herein are methods of treating a disease or condition in a subject in need thereof, comprising treating the disease or condition by administering, via inhalation, a therapeutically effective amount of the powdery pharmaceutical composition disclosed herein to the subject in need thereof.

In some aspects, administering may be conducted one, two, three, four, or more than four times per day. In some aspects, the disease or condition may be selected from the group consisting of: anaphylaxis, asthma, a cardiac disorder, an allergic reaction, cardiac arrest, superficial bleeding, an infection, hypertension (e.g., increased blood pressure), hypotension, septic shock, attention deficit hyperactivity disorder (ADHD), depression, a mood disorder, a psychiatric disorder, a type I diabetes, a type II diabetes, a type III diabetes, a type IIIc diabetes, a gestational diabetes, and any combination thereof. In some aspects, a powdery pharmaceutical composition may be administered as needed, or for about: one day, two days, three days, four days, five days, six days, a week, two weeks, three weeks, a month, two months, three months, four months, five months, six months, seven months, eight months, nine months, ten months, eleven months, a year, or chronically. In some aspects, an amount of the epinephrine or the pharmaceutically acceptable salt thereof may range from about 0.1 mg to about 0.5 mg. In some aspects, a second therapeutic or pharmaceutically acceptable salt thereof may be administered. In some aspects, a second therapeutic or a pharmaceutically acceptable salt thereof may be administered concurrently or consecutively. In some aspects, a second therapeutic or a pharmaceutically acceptable salt thereof may be comprised in a powdery pharmaceutical formulation. In some aspects, a second therapeutic or a pharmaceutically acceptable salt thereof may not be comprised in the powdery pharmaceutical formulation. In some aspects, a subject may be diagnosed with the disease or condition. In some aspects, the diagnosing may comprise employing an in vitro diagnostic. In some aspects, an in vitro diagnostic may be a companion diagnostic. In some aspects, a powdery pharmaceutical composition may be contained within a capsule, wherein the capsule is at least in part contained within an inhaler, and wherein the inhaler contains a sharp surface configured to puncture or slice the capsule, and wherein, prior to administrating, the inhaler is actuated such that the sharp surface punctures or slices the capsule. In some aspects, inhalation may be oral inhalation, intra nasal administration, or any combination thereof. In some aspects, in a human clinical trial, the powdery pharmaceutical composition, when inhaled into lungs, may provide in at least part of the humans in the clinical trial a time to peak plasma concentration (Tmax) of the epinephrine or the pharmaceutically acceptable salt thereof ranging from about 1 minute to about 10 minutes.

Also disclosed herein are methods of spray drying a liquid comprising: i) particles of epinephrine or a pharmaceutically acceptable salt thereof; ii) a coating material, wherein the coating material comprises a hydroxypropyl methylcellulose (HPMC), a hydroxypropyl methylcellulose acetate succinate (HPMCAS), a povidone, a copovidone, or any combination thereof; and iii) a solvent, wherein the particles of the epinephrine or the pharmaceutically acceptable salt thereof are at least partially dispersed in the liquid. In some aspects, the particles of the epinephrine or the pharmaceutically acceptable salt thereof at least partially dispersed in the liquid may have a particle diameter ranging from about 1 micrometer to about 5 micrometers. In some aspects, the spray drying may comprise i) atomizing liquid droplets comprising the epinephrine or the pharmaceutically acceptable salt thereof, the coating material, and the solvent, ii) drying the droplets to form substantially encapsulated particles, wherein the substantially encapsulated particles comprise the epinephrine or the pharmaceutically acceptable salt thereof substantially encapsulated by the coating material and iii) recovering the substantially encapsulated particles. In some aspects, the recovered particles of the epinephrine or the pharmaceutically acceptable salt thereof substantially encapsulated in the coating material may have a particle diameter ranging from about 1 micrometer to about 10 micrometers, or from about 1 micrometer to about 5 micrometers, as measured by a particle analyzer using laser diffraction.

Also disclosed herein are powdery pharmaceutical compositions, comprising: i) particles of a pharmaceutically acceptable excipient; and ii) particles comprising epinephrine or a pharmaceutically acceptable salt thereof, substantially encapsulated in a coating material, wherein the coating material comprises a hydroxypropyl methylcellulose (HPMC), a hydroxypropyl methylcellulose acetate succinate (HPMCAS), a povidone, a copovidone or any combination thereof, produced by a process comprising: a) mixing the particles comprising the epinephrine or the pharmaceutically acceptable salt thereof, the coating material, and a solvent; b) spray drying the mixed particles comprising epinephrine or the pharmaceutically acceptable salt thereof, the coating material, and the solvent to form the particles of ii) and blending the particles of i) and ii). In some aspects, the spray drying may comprise a) atomizing liquid droplets comprising the epinephrine or the pharmaceutically acceptable salt thereof, the coating material, and the solvent, b) drying the droplets to form substantially encapsulated particles, wherein the substantially encapsulated particles comprise the epinephrine or the pharmaceutically acceptable salt thereof substantially encapsulated by the coating material and c) recovering the substantially encapsulated particles.

Also disclosed herein are methods of making a powdery pharmaceutical composition, comprising blending: i) particles of a pharmaceutically acceptable excipient; and ii) a plurality of spray dried particles, each particle of the plurality of spray dried particles comprising epinephrine or a pharmaceutically acceptable salt thereof, substantially encapsulated in a coating material, wherein at least a portion of the plurality of spray dried particles comprising the epinephrine or the pharmaceutically acceptable salt thereof substantially encapsulated in the coating material have a particle diameter ranging from about 1 micrometer to about 10 micrometers, or from about 1 micrometer to about 5 micrometers, as measured by a particle analyzer using laser diffraction and, wherein the coating material comprises a hydroxypropyl methylcellulose (HPMC), a hydroxypropyl methylcellulose acetate succinate (HPMCAS), a povidone, a copovidone or any combination thereof.

Also disclosed herein are methods of increasing exercise performance in a subject in need thereof, comprising administering, via inhalation, a therapeutically effective amount of the powdery pharmaceutical composition disclosed herein to the subject in need thereof.

Also disclosed herein are methods treating a diabetes in a subject in need thereof, comprising administering, via inhalation, a therapeutically effective amount of the powdery pharmaceutical composition disclosed herein to the subject in need thereof.

### BRIEF DESCRIPTION OF THE DRAWINGS

FIG. 1A shows a dry powder inhaler device for delivery of a powdery pharmaceutical composition to the lung alveolar and FIG. 1B shows a nasal inhaled device for intranasal delivery of a powdery pharmaceutical composition to the lung alveolar.

FIG. 2 shows the method of use for the dry powder inhaler device for delivery of a powdery pharmaceutical composition.

FIG. 3 shows a spray drying manufacturing system comprising a closed spray dryer container which receives a solution comprising a drug dissolved or mixed in a suitable solvent. The system generates solid particles from the solution comprising the drug.

FIG. 4 shows a protective cap for a dry powder inhaler device.

FIG. 5 shows a rotatable mouthpiece of a dry powder inhaler device.

FIG. 6 shows a lower base chamber receptacle of a dry powder inhaler device.

FIG. 7 shows a lateral button operably connected to a sharp surface for use in a dry powder inhaler device for piecing a capsule containing a dry powdery pharmaceutical composition.

FIG. 8 shows a base plate of a dry powder inhaler device.

FIG. 9 shows a dry powder inhaler device for delivery of a powdery pharmaceutical composition to the lung alveolar.

FIG. 10 shows a spray drying manufacturing system comprising a closed spray drying chamber which receives a solution comprising an active ingredient, an encapsulating polymer, and a suitable solvent.

### DETAILED DESCRIPTION

### Overview

Delivering pharmaceutical compositions through oral ingestion of capsules or tablets may take a long time to dissolve and reach the blood stream. The absorption through stomach may take longer if fatty foods are eaten prior to ingestion of the capsule or tablet, further slowing down the process. By spray drying the pharmaceutical compositions and introducing them into the lungs via inhalation, the time needed for the pharmaceutical to reach the blood stream may be significantly reduced. In addition, the dosing level may also be reduced as compared to the oral tablet or capsule equivalent.

Provide herein among other things is the production of a pharmaceutical drug, epinephrine, which may also be known as adrenaline. Adrenaline may also be produced by the body. In some instances, the epinephrine drug may be delivered as a dry powder drug utilizing inhalation or intranasal administration as the route of administration. In some cases, epinephrine or a salt thereof may be microencapsulated.

Also provided herein is the production of a pharmaceutical drug, insulin. Insulin can be produced by the body. However, an autoimmune disease, type 1 diabetes, can cause insulin-producing cells in the pancreas to be destroyed. In another disease, type 2 diabetes, a subject may not make enough insulin or the body's cells may not respond to insulin. In some instances, type 1 and type 2 diabetes can be treated with an insulin. In some cases, an insulin disclosed herein can be administer as a dry powder drug via inhalation or intranasal administration. In some cases, an insulin herein can be microencapsulated.

Provided herein are pharmaceutical compositions, kits comprising pharmaceutical compositions, methods of treating disease, and methods of making compositions and kits described herein. Pharmaceutical drugs described herein may be produced employing various methods to synthesize, manipulate, and administer particles. In some aspects, the pharmaceutical compositions described herein are powdery pharmaceutical compositions.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various aspects may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining”, “measuring”, “evaluating”, “assessing,” “assaying,” and “analyzing” are often used interchangeably herein to refer to forms of measurement and include determining if an element may be present or not (for example, detection). These terms may include quantitative, qualitative or quantitative, and qualitative determinations. Assessing may be alternatively relative or absolute. “Detecting the presence of” includes determining the amount of something present, as well as determining whether it may be present or absent.

The terms “subject,” “individual,” or “patient” are often used interchangeably herein. A “subject” may be a biological entity containing expressed genetic materials. The biological entity may be a plant, animal, or microorganism, including, for example, bacteria, viruses, fungi, and protozoa. The subject may be tissues, cells and their progeny of a biological entity obtained in vivo or cultured in vitro. The subject may be a mammal. The mammal may be a human. The subject may be diagnosed or suspected of being at high risk for a disease. In some cases, the subject may not be necessarily diagnosed or suspected of being at high risk for the disease.

The term “substantially” or “essentially” may refer to a qualitative condition that exhibits an entire or nearly total range or degree of a feature or characteristic of interest. In some cases, substantially encapsulated may refer to near complete encapsulation of a substance or compound. For example, substantially encapsulated may comprise a particle that is at least about: 70%, 75%, 80%, 85%, 90%, 95%, 99%, or 100% encapsulated. In some cases, substantially may refer to at least about: 70%, 75%, 80%, 85%, 90%, 95%, 99%, or 100% of the total range or degree of a feature or characteristic of interest.

The term “at least partially” may refer to a qualitative condition that exhibits a partial range or degree of a feature or characteristic of interest. In some cases, at least partially encapsulated may refer to a partial encapsulation of a substance or compound. For example, at least partially encapsulated may comprise a particle that is at least about: 5%, 10%, 20%, 30%, 40%, 50%, 60%, 70%, 75%, 80%, 85%, 90%, 95%, 99%, or 100% encapsulated.

The term “in vivo” may be used to describe an event that takes place in a subject's body.

The term “ex vivo” may be used to describe an event that takes place outside of a subject's body. An “ex vivo” assay may not be performed on a subject. Rather, it may be performed upon a sample separate from a subject. An example of an “ex vivo” assay performed on a sample may be an “in vitro” assay.

The term “in vitro” may be used to describe an event that takes place contained in a container for holding laboratory reagent such that it may be separated from the living biological source organism from which the material may be obtained. In vitro assays may encompass cell-based assays in which cells alive or dead are employed. In vitro assays may also encompass a cell-free assay in which no intact cells are employed.

As used herein, the term ‘about’ a number may refer to that number plus or minus 10% of that number. The term ‘about’ a range may refer to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit may be achieved with the eradication or amelioration of one or more of the physiological symptoms associated with the underlying disorder such that an improvement may be observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

As used herein, the term “unit dose” or “dosage form” may be used interchangeably and may be meant to refer to pharmaceutical drug products in the form in which they are marketed for use, with a specific mixture of active ingredients and inactive components or excipients, in a particular configuration, and apportioned into a particular dose to be delivered. The term “unit dose” may also sometimes encompass non-reusable packaging, although the FDA distinguishes between unit dose “packaging” or “dispensing”. More than one unit dose may refer to distinct pharmaceutical drug products packaged together, or to a single pharmaceutical drug product containing multiple drugs and/or doses. The term “unit dose” may also sometimes refer to the particles comprising a phannaceutical composition, and to any mixtures involved. Types of unit doses may vary with the route of administration for drug delivery, and the substance(s) being delivered. A solid unit dose may be the solid form of a dose of a chemical compound used as a pharmaceutically acceptable drug or medication intended for administration or consumption.

As used herein, the term “fine particle fraction” or “fine particle fraction from the emitted dose” may refer to the mass of active agent having an aerodynamic diameter below about: 5 μm, 6 μm, 7 μm, 8 μm, 9 μm, or 10 μm. In some instances, the cutoff size may be less than or equal to an aerodynamic diameter of about 5 μm. In some instances, the cutoff size may be less than or equal to an aerodynamic diameter of about 6.4 μm. In some instances, the cutoff size may be less than or equal to an aerodynamic diameter of about 7 μm or about 8 μm. In some instances, the fine particle fraction may be often used to evaluate the efficiency of aerosol deaggregation. In some cases, fine particle fraction may be the mass of active agent having an aerodynamic diameter below about: 5 μm, 6 μm, 7 μm, 8 μm, 9 μm, or 10 μm as a percentage of an emitted dose mass. In some cases, fine particle fraction may be the mass of active agent having an aerodynamic diameter of more than about: 5 μm, 6 μm, 7 μm, 8 μm, 9 μm, or 10 μm as a percentage of an emitted dose mass. In some cases, fine particle fraction may be the mass of active agent having an aerodynamic diameter from about: 1 μm to about 10 μm, 1 μm to about 5 μm, 3 μm to about 6 μm, 4 μm to about 7 μm, 6 μm to about 12 μm or about 7 μm to about 10 μm as a percentage of an emitted dose mass. For example, a composition described herein may have a fine particle fraction of at least about: 20%, 25%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 75%, 80%, 85%, 90% or 95% upon aerosolization.

As used herein, a “dose” may refer to a measured quantity of a therapeutic agent to be taken at one time.

As used herein, “pharmaceutically acceptable salt” may refer to pharmaceutical drug molecules, which may be formed as a weak acid or base, chemically made into their salt forms, most frequently as the hydrochloride, sodium, or sulfate salts. Drug products synthesized as salts may enhance drug dissolution, boost absorption into the bloodstream, facilitate therapeutic effects, and increase its effectiveness. Pharmaceutically acceptable salts may also facilitate the development of controlled-release dosage forms, improve drug stability, extend shelf life, enhance targeted drug delivery, and improve drug effectiveness.

As used herein, “laser diffraction” may refer to a method for particle size analysis, which consists of scattering laser light off an assembly of particles, and collecting the scattered light using a spatial array of detectors. The signal from the detectors may be a pattern of scattered/diffracted light vs. angle. This pattern may result from many particles being illuminated by the laser light source at the same time, where all of their individual scattered/diffracted light rays mix together at each detector element.

As used herein, “particle size analyzer” may refer to an instrument for particle size analysis, particle size measurement, or simply particle sizing.

As used herein, “particle size analysis” may refer to the collective name of the technical procedures, or laboratory techniques which determines the size range, and/or the average (mean), median or mode size of the particles, or droplets in a powder or liquid sample.

As used herein, “time to peak plasma concentration” may refer to the time required for a drug to reach peak concentration in plasma. Peak concentration in plasma may be usually defined as the plasma concentration that a drug achieves in a specified compartment or test area of the body after the drug has been administered and before the administration of a second dose.

As used herein, “HPLC” may refer to high-performance liquid chromatography (formerly referred to as high-pressure liquid chromatography), which is a technique in analytical chemistry used to separate, identify, and quantify each component in a mixture. HPLC may be a common technique used in pharmaceutical development, as it may be a method to ensure product purity.

As used herein, the terms “effective amount” or “therapeutically effective amount” of a drug used to treat a disease may be an amount that may reduce the severity of a disease, reduce the severity of one or more symptoms associated with the disease or its treatment, or delay the onset of more serious symptoms or a more serious disease that may occur with some frequency following the treated condition. An “effective amount” may be determined empirically and in a routine manner, in relation to the stated purpose.

The section headings used herein are for organizational purposes only and are not to be construed as limiting the subject matter described.

### Pharmaceutical Compositions

Disclosed herein are devices, systems and methods for producing, packaging, and delivering stable powdery pharmaceutical compositions to the lungs via intranasal inhalation. In some aspects, the pharmaceutical compositions may be spray dried. In those aspects, the addition of an excipient carrier product to the active pharmaceutical powders prior to encapsulation may improve its stability and effective solubility.

Advancements in engineering and process automation allow for enhanced methods of manufacturing to achieve consistent active ingredient particle size in the 1.0-10.0 micrometer size, which contributes to proper absorption in the lungs. A drug may be processed using spray drying technology to control the particle size and particle size distribution. Spray drying may produce active ingredient particle size in the 1.0-10.0-micrometer range. This particle size may be needed when a drug is administered by inhalation or by an intranasal route of administration for absorption into the lung alveolar. In some instances, the particle may be microencapsulated to enhance bioavailability. This route of administration may result in a rapid introduction of the drug into the blood stream and may require lower dosing when compared to oral intake of a capsule or tablet. In some instances, introducing encapsulated epinephrine into the lungs via inhalation, may allow epinephrine to reach the blood stream within 5 minutes. In some instances, introducing encapsulated insulin into the lungs via inhalation, may allow insulin to reach the blood stream within 5 minutes.

In some aspects the compositions may comprise one or more of: an active ingredient or salts, excipients, and inactive ingredients. In some cases, a pharmaceutical composition may comprise particles. In some cases, particles may comprise an excipient (e.g., a pharmaceutically acceptable excipient), an active ingredient, an encapsulated active ingredient or any combination thereof. In some cases, the compositions may comprise a pharmaceutical composition. In some instances, a composition may comprise particles of a pharmaceutically acceptable excipient. In some instances, a composition may comprise particles of an active ingredient. In some instances, a composition may comprise encapsulated particles of an active ingredient, for example HPMCAS encapsulated epinephrine or HPMCAS encapsulated insulin.

As used herein, “coating material” may refer to a material added via a pharmaceutical coating process by which an essentially dry, outer layer of coating material may be applied to the surface of a dosage form. Coating dosage forms may be used to improve stability (light protection, moisture and gas barrier), facilitate administration, or modify the drug release behavior from the dosage form. The coating materials may be used to enable the immediate release of the drug, delay the release of the drug (such as in enteric coatings), or sustain the release of the drug from the dosage form over extended periods of time. Coating materials may include film coating formulations, which usually contain a polymer, a plasticizer, a colorant, opacifier, a solvent, and a vehicle. In some cases, a coating material may refer to the coating material used in the coating of a particle of an active ingredient to create an encapsulated particle.

In some instances, the spray dried particles described above and herein can be encapsulated by one or more additional coating materials. In some cases, the coating materials are the same coating material. In some cases, the coating materials are different coating materials. In some instances, the powdery composition as described above may be a pharmaceutical composition optionally in unit dose form.

In some aspects, a composition may comprise a mixture of particles described herein. In some aspects, the particles may be mixed in a substantially homogenous mixture. In some instances, at least a portion of the particles of the pharmaceutically acceptable excipient may have a particle diameter ranging from about 50 micrometers to about 200 micrometers, as measured by a particle size analyzer using laser diffraction. In some cases, at least a portion of the active ingredient particles may have a particle diameter ranging from about 500 nanometers to about 15 micrometers, or about 1 micrometer to about 20 micrometers, as measured by a particle size analyzer using laser diffraction. In some instances, in a human clinical trial, the powdery pharmaceutical composition, when inhaled into the lungs, may provide in at least part of the humans in the clinical trial a time to peak plasma concentration (Tmax) of the active ingredient or the salt thereof ranging from about 1 minute to about one hour, or from about 1 minute to about ten minutes. In some aspects, the Tmax of the active ingredient or the salt thereof ranging from about 1 min to about 5 min, about 1 min to about 10 min, about 1 min to about 20 min, about 1 min to about 25 min, about 1 min to about 30 min, about 1 min to about 40 min, about 1 min to about 50 min, about 1 min to about 60 min, about 5 min to about 10 min, about 5 min to about 20 min, about 5 min to about 25 min, about 5 min to about 30 min, about 5 min to about 40 min, about 5 min to about 50 min, about 5 min to about 60 min, about 10 min to about 20 min, about 10 min to about 25 min, about 10 min to about 30 min, about 10 min to about 40 min, about 10 min to about 50 min, about 10 min to about 60 min, about 20 min to about 25 min, about 20 min to about 30 min, about 20 min to about 40 min, about 20 min to about 50 min, about 20 min to about 60 min, about 25 min to about 30 min, about 25 min to about 40 min, about 25 min to about 50 min, about 25 min to about 60 min, about 30 min to about 40 min, about 30 min to about 50 min, about 30 min to about 60 min, about 40 min to about 50 min, about 40 min to about 60 min, or about 50 min to about 60 min.

In some aspects, when inhaled into the lungs in a human clinical trial, the powdery pharmaceutical composition operates mechanistically such that in at least a portion of the humans in the clinical trial, a majority of the particles of the pharmaceutically acceptable excipient deposit onto the oropharynx.

In some aspects, the weigh to weight ratio of: a) the particles of the pharmaceutically acceptable excipient and b) the active ingredient particles ranges from about 1:1 to about 10000:1. In some aspects, the weight to weight ratio of: a) the particles of the pharmaceutically acceptable excipient and b) the active ingredient particles ranges from about 1:1 to about 20:1, about 1:1 to about 15:1, about 1:1 to about 10:1, about 1:1 to about 5:1, about 1:1 to about 2:1, about 2:1 to about 20:1, about 2:1 to about 15:1, about 2:1 to about 10:1, about 2:1 to about 5:1, about 5:1 to about 20:1, about 5:1 to about 15:1, about 5:1 to about 10:1, about 10:1 to about 15:1, about 10:1 to about 20:1, about 15:1 to about 20:1, about 18:1 to about 25:1, or about 25:1 to about 30:1. In some aspects, the weight to weight ratio of: a) the particles of the pharmaceutically acceptable excipient and b) the active ingredient particles may be about: 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, or 30:1 In some aspects, the weight to weight ratio of: a) the particles of the pharmaceutically acceptable excipient and b) the active ingredient particles ranges from about 1:1 to about 1:10, about 1:1 to about 1:8, about 1:1 to about 1:5, about 1:1 to about 1:2, about 1:2 to about 1:10, about 1:2 to about 1:8, about 1:2 to about 1:5, about 1:5 to about 1:10, about 1:5 to about 1:8, about 1:8 to about 1:10.

In some aspects, at least a portion of the particles of the pharmaceutical excipient and the active ingredient particles may not be covalently bound to each other.

### Active Ingredients and Pharmaceutically Acceptable Salts

An active pharmaceutical ingredient may be any substance or mixture of substances intended to be used in the manufacture of a drug (medicinal) product and that, when used in the production of a drug, becomes an active ingredient of the drug product. Such substances may be intended to furnish pharmacological activity or other direct effect in the diagnosis, cure, mitigation, treatment, or prevention of disease or to affect the structure or function of the body. In some aspects, an active pharmaceutical ingredient or salt thereof may be formulated as a powder. For example, an epinephrine or an insulin disclosed herein may be formulated as a powder using the methods described herein. In some cases, an active ingredient may comprise a pharmaceutical compound. In some cases, a pharmaceutical compound may comprise an active ingredient.

In some aspects, the active pharmaceutical ingredients may comprise alpha- and beta-adrenergic agonists (e.g., sympathomimetic agent/drug) or pharmaceutically acceptable salts thereof. In some aspects, the alpha- and beta-adrenergic agonists may be epinephrine (e.g., adrenaline) or a pharmaceutically acceptable salt thereof. In some cases, the active pharmaceutical ingredient may comprise an encapsulated epinephrine or a pharmaceutically acceptable salt thereof. In some cases, an epinephrine or a salt thereof can comprise racepinephrine. In some cases, epinephrine can comprise D-epinephrine, L-epinephrine, or a mixture thereof. In some aspects, the sympathomimetic agents may comprise norepinephrine, dopamine, albuterol, salmeterol, formeterol, terbutaline, conadine, guanfacine, dobutamine, fenoldopam, isoproterenol, methyldopa, midodrine, mirabegrown, oxymetazoline, phenylephrine, or a salt of any of these. In some cases, an epinephrine may comprise epinephrine hydrochloride, epinephrine bitartrate, or epinephrine borate. In some cases, epinephrine may be a sympathomimetic catecholamine. In some cases, the chemical name of epinephrine is: 1,2-Benzenediol, 4-[(1R)-1-hydroxy-2-(methylamino)ethyl]-, or (−)-3,4-Dihydroxy-α-[2(methylamino)ethyl]benzyl alcohol. In some cases, the molecular weight of epinephrine may be 183.2. In some cases, the chemical structure of epinephrine is C₉H₁₃NO₃ and is represented by Formula I.

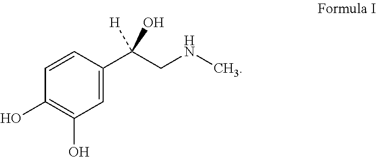 [SMILES: CNC[C@H](O)c1ccc(O)c(O)c1]

The disclosure herein includes all such possible isomers, as well as their racemic and optically pure forms of an epinephrine or a salt thereof or any compound disclosed herein. A stereoisomer can refer to a compound made up of the same atoms bonded by the same bonds but having different three-dimensional structures, which are not interchangeable. The present disclosure contemplates various stereoisomers and mixtures thereof and includes enantiomers, which can refer to two stereoisomers whose molecules are nonsuperimposeable mirror images of one another. Optically active (+) and (−), (R) and (S), or (D) and (L) isomers may be prepared using chiral synthons or chiral reagents, or resolved using conventional techniques, for example, chromatography, chiral chromatography, HPLC, chiral HPLC, and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

In some embodiments, a compound or a salt thereof (e.g., an epinephrine) may comprise an enantiomerically pure form. In some examples, the compound or salt thereof disclosed herein can have an enantiomeric excess greater than about or equal to: 5%, 10%, 15%, 20%, 25%, 30%, 35%, 40%, 45%, 50%, 60%, 70%, 80%, 90%, 95%, 96%, 97%, 98%, or 99%. A compound or a salt thereof may be dosed in their diasteriomerically pure form. In some examples, the compound or a salt thereof can have a diasteriomeric excess greater than about or equal to: 5%, 10%, 15%, 20%, 25%, 30%, 35%, 40%, 45%, 50%, 55%, 60%, 65%, 70%, 75%, 80%, 90%, 95% or 99%. In some cases, percent enantiomeric excess can be defined as: % e=([F_{R}−F_{S}]×100), wherein FR is the mole fraction of the compound with an R stereocenter and FS is the mole fraction of the compound with an S stereocenter and the two vertical lines indicate taking the absolute value of the difference.

In some aspects, an active pharmaceutical ingredient can comprise an insulin. In some cases, an insulin can comprise a rapid-acting insulin, a short-acting insulin, an intermediate-acting insulin, a long-acting insulin, an ultra-long acting insulin, or a mixture thereof. In some cases, an insulin can comprise an insulin analog. In some cases, an insulin analog can comprise an insulin aspart, an insulin lispro, an insulin glulisine, or a mixture thereof. In some cases, an insulin can comprise a human insulin, an Insulin Human Isophane (NPH) human insulin, or a mixture thereof. In some cases, an insulin can comprise a novolin, a velosulin, or a mixture thereof. In some cases, a long acting insulin can comprise an insulin glargine, an insulin degludec, an insulin detemir or a mixture thereof. In some cases, a long acting insulin can comprise an insulin analog. In some cases, an insulin can comprise insulin glargine, insulin degludec, or any combination thereof. In some cases, an insulin can comprise a pre-mixed insulin. In some cases, a pre-mixed insulin can comprise a mixture of an intermediate-acting and short-acting insulin. In some cases, an insulin can comprise a derivative of insulin. In some cases, a derivative of a compound or molecule disclosed herein, can refer to a substance related structurally to the compound or molecule disclosed herein. A derivative can be made from a structurally-related parent compound in one or more steps. In some cases, a derivative can comprise one or more amino acid substitutions to the parent molecule. In some cases, the general physical and chemical properties of a derivative can be similar to a parent compound or molecule.

In some aspects, active pharmaceutical ingredients or salts may comprise an antibiotic, an antiviral, an antiparasitic, a diuretic, a blood pressure medication, a phosphodiesterase inhibitor, a pharmaceutically acceptable salt of any of these, or any combination thereof. In some cases, an active pharmaceutical ingredient may comprise a beta-blocker, an ACE inhibitor, an angiotensin II receptor blocker, a calcium channel blocker, an alpha blocker, a cancer chemotherapeutic, a steroid, an immunomodulator, a pharmaceutically acceptable salt of any of these, or any combination thereof. In some cases, an antibiotic may comprise a penicillin, a cephalosporin, a tetracycline, an aminoglycoside, a macrolide, clindamycin, a sulfonamide, a trimethoprim, a metronidazole, a quinolone, or a nitrofurantoin. An antiviral may comprise an acyclovir, peramivir, zanamivir, oseltamivir phosphate, remdesivir, balozavir marboxil, a salt of any of these or any combination thereof. In some cases, an active pharmaceutical ingredient or salt thereof may comprise a potassium channel blocker such as dalfampridine or a salt thereof. In some cases, an active pharmaceutical ingredient or salt thereof may comprise levodopa, carbidopa, or a salt thereof. In some cases, an active pharmaceutical ingredient or salt thereof may comprise an antibody such as rHlgM22. In some cases, an active pharmaceutical ingredient or salt thereof may comprise a biologic such as GGF2 (Cimaglermin alfa). In some cases, an active pharmaceutical ingredient or salt thereof may comprise zolmitriptan or a salt thereof.

In some aspects, active pharmaceutical ingredients or salts thereof may comprise a cannabinoid such as a tetrahydrocannabinol, a cannabidiol, a pharmaceutically acceptable salt of any of these, or any combination thereof.

In some aspects, the composition may further comprise: another set of active pharmaceutical ingredients or salts thereof. For example, a second, third, or fourth different set of active pharmaceutical ingredients. In some aspects, the additional pharmaceutical ingredients or salts thereof may be administered in parallel or consecutively to enhance the efficacy of the first set of active pharmaceutical ingredients or salts.

In some aspects, a composition may further comprise: an additional set of active pharmaceutical ingredients or salts thereof which may be administered in parallel or consecutively to enhance the efficacy of epinephrine or a salt thereof. In some aspects, a second different set of active pharmaceutical ingredients or salts may be administered in parallel or consecutively to enhance the efficacy of epinephrine or a salt thereof. In some aspects, a composition may comprise two or more different sets of active pharmaceutical ingredients or salt thereof which may be administered in parallel or consecutively to enhance the efficacy of epinephrine or a salt thereof.

In some aspects, a composition may further comprise: an additional set of active pharmaceutical ingredients or salts thereof which may be administered in parallel or consecutively to enhance the efficacy of an insulin. In some aspects, a second different set of active pharmaceutical ingredients or salts may be administered in parallel or consecutively to enhance the efficacy of an insulin. In some aspects, a composition may comprise two or more different sets of active pharmaceutical ingredients or salt thereof which may be administered in parallel or consecutively to enhance the efficacy of an insulin or a salt thereof.

In some aspects the first set of active pharmaceutical ingredients or salts may be administered in parallel or consecutively with a second different set of active pharmaceutical ingredients. In some cases, the pharmaceutical ingredients may comprise nitrates, nitric oxide, nitric oxide generating components, nitrite salts, nitrate salts, sodium nitrates, potassium nitrates, vitamin C, ascorbic acid, L-arginine, L-citrulline, vitamin B12, magnesium ascorbate, sodium ascorbate, potassium ascorbate, antihypertensive agents, diuretics, salts thereof, or any combination thereof.

In some aspects, the pharmaceutical ingredients may comprise phosphodiesterase 5 inhibitors such as sildenafil, tadalafil, vardenafil, avanafil, mirodenafil, udenafil, iodenafil, derivatives of these, salts of these, or any combinations thereof.

In some cases, the pharmaceutical ingredients may comprise beta blockers (β-blockers), calcium blockers, angiotensin converting enzyme inhibitors, angiotensin receptor blockers, Nebivolol, CYP3A4 inhibitors, ketoconazole (Nizoral), itraconazole (Sporanox), erythromycin, saquinavir, clarithromycin, HIV protease inhibitors, alpha-adrenergic blocking agents (α-blockers), salts thereof, or any combination thereof.

In some aspects, a second different set of active pharmaceutical ingredients or salts may not be comprised in the powdery pharmaceutical composition. In some aspects, a second different set of active pharmaceutical ingredients or salts not comprised in the powdery pharmaceutical composition may be administered concurrently, in parallel, or consecutively.

In some aspects, the pharmaceutical composition has metabolites that may be pharmacologically active, retaining, at least partially, the potency of the parent drug or the parent pharmaceutical component.

In some aspects, the pharmaceutical composition comprising the salt of the pharmaceutically active ingredient, wherein the salt comprises an organic salt, an inorganic salt, or any combination thereof. In some cases, an organic salt may comprise a phosphinate (e.g., sodium hypophosphite), a hydrazinium salt, a urate, a diazonium salt, an oxalate salt, a tartrate, a choline chloride. An example of an inorganic salt may be sodium chloride, calcium chloride, magnesium chloride, sodium bicarbonate, potassium chloride, sodium sulfate, calcium carbonate, calcium phosphate, or any combination thereof.

In some aspects, the pharmaceutical composition comprising the salt of the pharmaceutically active ingredient, wherein the salt comprises an HCl salt, an ascorbic acid salt, a mandelic acid salt, an aspartic acid salt, a carbonic acid salt, a citric acid salt, a formic acid salt, a glutamic acid salt, a lactic acid salt, a lauric acid salt, a maleic acid salt, a borate salt, a bitartrate salt, a palmitic acid salt, a phosphoric acid salt, or any combination thereof.

In some aspects, the pharmaceutically acceptable salts include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; inorganic acid salts such as hydrochloride, hydrobromide, phosphate, sulphate and the like; organic acid salts such as citrate, lactate, tartrate, maleate, fumarate, mandelate, acetate, dichloroacetate, trifluoroacetate, oxalate, formate and the like; sulfonates such as methanesulfonate, benzenesulfonate, p-toluenesulfonate and the like; and amino acid salts such as arginate, asparginate, glutamate and the like.

### Excipients and Other Non-Active Ingredients

In some aspects, the pharmaceutical composition comprises pharmaceutically acceptable excipients. As used herein, “excipient” may refer to a substance formulated alongside the active ingredient of a medication, included for the purpose of long-term stabilization, bulking up solid formulations that contain potent active ingredients in small amounts, and/or to confer a therapeutic enhancement on the active ingredient(s) in the final dosage form. Excipients may facilitate drug absorption, reduce viscosity, or enhance solubility. Excipients may also facilitate the handling of the active ingredients, improve in vitro stability, and/or extend pharmaceutical product shelf life. Excipient selection may vary with the route of administration for drug delivery, the unit dose, as well as the active ingredients comprising the composition.

In some aspects, an excipient may comprise anhydrous calcium phosphate, dihydrate calcium phosphate, hydroxypropyl methylcellulose, croscarmellose sodium, GMO-free croscarmellose sodium, carbomers, magnesium aluminometasilicate, mannitol, povidone (PVP), crospovidone, sorbitol, dimethicone, sodium stearyl fumarate, sodium starch glycollate, hydroxypropylcellulose, native corn starch, modified corn starch, carrageenan, alginates, silicon dioxide, microcrystalline cellulose, carboxymethylcellulose sodium, alginates, carboxymethylcellulose (CMC), sodium carboxymethylcellulose (Na CMC), carbomers, natural gums, sorbitol, maltitol, glucose syrup, silicones, carbomers, fatty alcohols, alcohols, carbohydrates, petrolatum derivatives, butters, waxes, DMSO, esters, fatty acids, oil-in-water (O/W) emulsifiers, water-in-oil (W/O) emulsifiers, silicas, fumed silicas, polysorbates, isopropyl myristate, cellulosic derivates, xanthan gum, propylenglycol, noveon AA-1 polycarbophyl, dimethyl isosorbate, polysilicone elastomer 1100, polysilicone elastomer 1148P, preservatives, flavors, colors, functional coatings, aesthetic coatings, a pharmaceutically acceptable salt of any of these, or any combination thereof.

In some cases, a pharmaceutically acceptable excipient may comprise acacia, acesulfame potassium, acetic acid, glacial, acetone, acetyl tributyl citrate, acetyl triethyl citrate, agar, albumin, alcohol, alginic acid, aliphatic polyesters, alitame, almond oil, alpha tocopherol, aluminum hydroxide adjuvant, aluminum oxide, aluminum phosphate adjuvant, aluminum stearate, ammonia solution, ammonium alginate, ascorbic acid, ascorbyl palmitate, aspartame, attapulgite, bentonite, benzalkonium chloride, benzethonium chloride, benzoic acid, benzyl alcohol, benzyl benzoate, boric acid, bronopol, butylated hydroxyanisole, butylated hydroxytoluene, butylparaben, calcium alginate, calcium carbonate, calcium phosphate, dibasic anhydrous, calcium phosphate, dibasic dihydrate, calcium phosphate, tribasic, calcium stearate, calcium sulfate, canola oil, carbomer, carbon dioxide, carboxymethylcellulose calcium, carboxymethylcellulose sodium, carrageenan, castor oil, castor oil, hydrogenated, cellulose (e.g., microcrystalline, powdered, silicified microcrystalline, acetate, acetate phthalate) ceratonia, cetostearyl alcohol, cetrimide, cetyl alcohol, cetylpyridinium chloride, chitosan, chlorhexidine, chlorobutanol, chlorocresol, chlorodifluoroethane, chlorofluorocarbons, chloroxylenol, cholesterol, citric acid monohydrate, colloidal silicon dioxide, coloring agents, copovidone, corn oil, cottonseed oil, cresol, croscarmellose sodium, crospovidone, cyclodextrins, cyclomethicone, denatonium benzoate, dextrates, dextrin, dextrose, dibutyl phthalate, dibutyl sebacate, diethanolamine, diethyl phthalate, difluoroethane, dimethicone, dimethyl ether, dimethyl phthalate, dimethyl sulfoxide, dimethylacetamide, disodium edetate, docusate sodium, edetic acid, erythorbic acid, erythritol, ethyl acetate, ethyl lactate, ethyl maltol, ethyl oleate, ethyl vanillin, ethylcellulose, ethylene glycol palmitostearate, ethylene vinyl acetate, ethylparaben, fructose, fumaric acid, gelatin, glucose, glycerin, glyceryl behenate, glyceryl monooleate, glyceryl monostearate, glyceryl palmitostearate, glycofurol, guar gum, hectorite, heptafluoropropane, hexetidine, hydrocarbons, hydrochloric acid, hydroxyethyl cellulose, hydroxyethylmethyl cellulose, hydroxypropyl cellulose, hydroxypropyl cellulose, low-substituted, hydroxypropyl starch, hypromellose, hypromellose acetate succinate, hypromellose phthalate, honey, imidurea, inulin, iron oxides, isomalt, isopropyl alcohol, isopropyl myristate, isopropyl palmitate, kaolin, lactic acid, lactitol, lactose, anhydrous, lactose, monohydrate, lactose, spray-dried, lanolin, lanolin alcohols, lanolin, hydrous, lauric acid, lecithin, leucine, linoleic acid, macrogol hydroxystearate, magnesium aluminum silicate, magnesium carbonate, magnesium oxide, magnesium silicate, magnesium stearate, magnesium trisilicate, malic acid, maltitol, maltitol solution, maltodextrin, maltol, maltose, mannitol, medium-chain triglycerides, meglumine, menthol, methylcellulose, methylparaben, mineral oil, mineral oil and lanolin alcohols, monoethanolamine, monosodium glutamate, monothioglycerol, myristic acid, neohesperidin dihydrochalcone, nitrogen, nitrous oxide, octyldodecanol, oleic acid, oleyl alcohol, olive oil, palmitic acid, paraffin, peanut oil, pectin, petrolatum, petrolatum and lanolin alcohols, phenol, phenoxyethanol, phenylethyl alcohol, phenylmercuric acetate, phenylmercuric borate, phenylmercuric nitrate, phosphoric acid, polacrilin potassium, poloxamer, polycarbophil, polydextrose, polyethylene glycol, polyethylene oxide, polymethacrylates, poly(methyl vinyl ether/maleic anhydride), polyoxyethylene alkyl ethers, polyoxyethylene castor oil derivatives, polyoxyethylene sorbitan fatty acid esters, polyoxyethylene stearates, polyvinyl acetate phthalate, polyvinyl alcohol, potassium alginate, potassium benzoate, potassium bicarbonate, potassium chloride, potassium citrate, potassium hydroxide, potassium metabisulfite, potassium sorbate, povidone, propionic acid, propyl gallate, propylene carbonate, propylene glycol, propylene glycol alginate, propylparaben, 2-pyrrolidone, raffinose, saccharin, saccharin sodium, saponite, sesame oil, shellac, simethicone, sodium acetate, sodium alginate, sodium ascorbate, sodium benzoate, sodium bicarbonate, sodium borate, sodium chloride, sodium citrate dihydrate, sodium cyclamate, sodium hyaluronate, sodium hydroxide, sodium lactate, sodium lauryl sulfate, sodium metabisulfite, sodium phosphate, dibasic, sodium phosphate, monobasic, sodium propionate, sodium starch glycolate, sodium stearyl fumarate, sodium sulfite, sorbic acid, sorbitan esters (sorbitan fatty acid esters), sorbitol, soybean oil, starch, starch (e.g., pregelatinized, sterilizable maize), stearic acid, stearyl alcohol, sucralose, sucrose, sugar, compressible, sugar, confectioner's, sugar spheres, sulfobutylether b-cyclodextrin, sulfuric acid, sunflower oil, suppository bases, hard fat, talc, tartaric acid, tetrafluoroethane, thaumatin, thimerosal, thymol, titanium dioxide, tragacanth, trehalose, triacetin, tributyl citrate, triethanolamine, triethyl citrate, vanillin, vegetable oil, hydrogenated, water, wax, anionic emulsifying, wax (e.g., carnauba, cetyl esters, microcrystalline, nonionic emulsifying, white, yellow), xanthan gum, xylitol, zein, zinc acetate, zinc stearate, or any combination thereof.

In some aspects, a pharmaceutically acceptable excipient may comprise a carbohydrate, an alginate, povidone, a carbomer, a flavor, a natural gum, a silicone, an alcohol, a butter, a wax, a fatty acid, a preservative, a pharmaceutically acceptable salt of any of these, or any combination thereof. In some aspects, a pharmaceutically acceptable excipient may comprise a carbohydrate. In some instances, the carbohydrate may comprise lactose, microcrystalline cellulose, cellulose, mannitol, sorbitol, starch, starch glycolate, hydroxypropyl methylcellulose, hydroxypropyl methylcellulose acetate succinate, a cyclodextrin, maltodextrin, croscarmellose sodium, corn starch, carrageenan, sorbitol, maltitol, glucose, a pharmaceutically acceptable salt of any of these, or any combination thereof. In some aspects, a pharmaceutically acceptable excipient may comprise lactose. In some instances, lactose may comprise milled lactose, sieved lactose, micronized lactose, spray dried lactose, at least substantially anhydrous lactose, monohydrate lactose, or a combination thereof.

In some aspects, the active ingredient or pharmaceutically acceptable salt thereof may be contained at least in part within an excipient. In some aspects, the active ingredient or pharmaceutically acceptable salt thereof may be contained at least in part in an excipient. In some aspects, the active ingredient may be contained within a pore of an excipient. The “pore” of the excipient may refer to excipient particles that have been engineered to have open or closed pore structures. Porous excipient particles may be carriers of pharmaceutically active ingredients. Porous excipient particles may have a large surface area, stable structure, adjustable pore sizes, tunable dissolution, diffusion, or distribution, and well-defined surface properties. Porous excipient particles may facilitate sustained-release unit doses.

In some aspects, in addition to the active pharmaceutical ingredients or salts thereof, the compositions may further comprise inactive ingredients selected from the group consisting of microcrystalline cellulose, anhydrous dibasic calcium phosphate, croscarmellose sodium, magnesium stearate, hypromellose, titanium dioxide, lactose, triacetin, mannitol, xylitol, sorbitol, sugar alcohols, cellulose, cellulose esters, cellulose ethers, modified celluloses, starch, modified starches, polysaccharides, oligosaccharides, disaccharides, saccharides, gelatin, polyvinylpyrrolidone, polyethylene glycol, binders, flavorants, colorants, FD & C Blue #2 aluminum lake, magnesium stearate, antiadherent agents, stearate salts, sweeteners, silica, lubricants, or any combination thereof.

### Method of Making the Powdery Pharmaceutical Composition

In some cases, methods of making a pharmaceutical composition may comprise creating particles by the methods described herein. In some cases, particles may comprise an excipient (e.g., a pharmaceutically acceptable excipient), an active ingredient, or both. In some aspects, a method of making a powdery pharmaceutical composition, may comprise mixing, in a mixer, particles of a pharmaceutically acceptable excipient; and particles comprising an active ingredient or a pharmaceutically acceptable salt thereof. In some cases, the particles comprising an active ingredient may be microencapsulated. For example, particles of epinephrine may be microencapsulated with a HPMC or HPMCAS coating. In another example, particles of insulin may be microencapsulated with a HPMC or a HPMCAS coating or a coating with two layers, the first layer comprising HPMC and the second layer comprising HPMCAS. In some instances, at least a portion of the particles of the pharmaceutically acceptable excipient may have a particle diameter ranging from about 50 micrometers to about 200 micrometers, as measured by a particle size analyzer using laser diffraction. In some cases, particles comprising the active ingredient may have a particle diameter ranging from about 500 nanometers to about 15 micrometers, or 1 micrometer to about 20 micrometers, as measured by a particle size analyzer using laser diffraction.

In some aspects, a method of making the powdery pharmaceutical composition may comprise particles wherein at least a portion of the particles of the active ingredient or a pharmaceutically acceptable salt thereof may be made by a spray drying process.

In some aspects, the spray drying process may comprise: atomizing liquid droplets comprising the active ingredient or the pharmaceutically acceptable salt thereof, drying the droplets from particles, recovering the particles, or any combination thereof.

In some aspects, a spray drying manufacturing system may comprise a closed spray dryer container which receives the solution comprising a drug dissolved or mixed in a suitable solvent (aqueous or solvent based). In some cases, a solvent may comprise alcohol, ethanol, dimethylformamide (DMF), dimethyl sulfoxide (DMSO), a polar organic solvent, an organic solvent, or any combination thereof. In some cases, a solvent can be a sugar solution such as a sugar solution of trehalose. In some cases, a solvent may comprise water or a mixture of water and another solvent disclosed herein. In some aspects, the solution then enters the particle formation chamber which may be connected to an atomizer located at the top of the chamber. In some aspects, the atomizer may be a two component or rotary nozzle type that distributes the solution into fine droplets controlled by the atomizer pressure. In some aspects, this atomization gas may be an inert gas. As used herein, “inert gas” may refer to a non-reactive gas, or a gas that does not undergo chemical reactions under a set of given conditions. Inert gases may be generally used to avoid unwanted chemical reactions degrading a sample, or to prevent bacterial growth. These undesirable chemical reactions may often be oxidation and hydrolysis reactions with the oxygen and moisture in air. The term “inert gas” may be context-dependent because several of the noble gases, which have been historically referred to as the inert gases, may be made to react under certain conditions. In some aspects, inert gas may be air, nitrogen, carbon dioxide or any combination thereof. In some aspects, the atomized droplets go through a hot gas drying chamber to produce uniform fine particles that maintain a tight particle size distribution following liquid evaporation. In some cases, the solid particle forms and falls to the bottom of the drying chamber. In some instances, the balance between temperature, flow rate, and droplet size may controls the drying process. In some aspects, the powder may be recovered from the exhaust gas using a cyclone or a bag filter. In some aspects, particle size may be validated by a Malvern particle analyzer prior to blending with an excipient carrier. In some aspects, the active powder (e.g., the powdery pharmaceutical composition) may be blended with an excipient carrier (lactose) product in a Patterson Kelly (PK Blender) and the blended powder may be fed to a hopper. In some aspects, from the hopper, the dry powder may be placed into a Size 3 Hypromellose capsule, by a Bosch Encapsulator machine. In some cases, the dry powder may be placed into any capsule of any size. For example, the dry powder may be placed into a size 000, 00, 0, 1, 2, 3, or a 4 size capsule.

The moisture level of the powder after spray drying may be below about 10%. In some aspects, the moisture level may be below about 15%, about 14%, about 13%, about 12%, about 11%, about 10%, about 9%, about 8%, about 7%, about 6%, about 5%, about 4%, about 3%, about 2%, or about 1%.

### Microencapsulation

In some aspects, encapsulation may comprise microencapsulation.

Microencapsulation may be a process in which a microcapsule may be created as a small sphere or multi-sphere in one core with a matrix wall around it. The pharmaceutical ingredient inside the microcapsule may be called a fill. In some cases, a fill may be a liquid, an oil, a solid or any combination thereof. The wall around the fill (“or core”) may be referred to as a shell, a coating, or a membrane. In some cases, a microcapsule can comprise more than one shell. For example, a microcapsule can comprise 2, 3, 4, 5, 6, 7, 8, 9, or more shells. Microcapsules may have a diameter as small as 1.0 micron in size to about 10.0 micron in size or about 1.0 micron to about 5.0 microns in size. In some cases, the small size may provide a pharmaceutical ingredient a large surface area to be available for absorption, release, transfer, or any combination thereof.

In some cases, a microencapsulated shell can comprise a coating to allow delivery to another part of the body or to the area where the composition was originally delivered. For example, a microencapsulated shell can comprise an enteric coating, a time release coating, a pH dependent coating, a delayed release coating, an extended-release coating, or a combination thereof. In some cases, the microencapsulated shell can be thicker to deliver the cell further into the body. In some cases, the microencapsulated shell can be a mixture of thicker and thinner shell sizes to have a controlled release. In some cases, the microencapsulated shell can comprise a controlled released coating or a mixture of controlled release coatings to provide dosing throughout different parts of the body. For example, microencapsulated cells can be configured to release in the small intestine and in the large intestine.

In some embodiments, a microencapsulated particle can be configured (for example, with one or more layers of an enteric coating) to at least partially release an active ingredient in: the mouth, the esophagus, the small intestine, the duodenum, the jejunum, the ileum, the cecum, the colon, the ascending colon, the traverse colon, the descending colon, the sigmoid colon, the rectum, the anus, or any combination thereof.

In some cases, microencapsulation may at least partially prevent inhalation of an active ingredient comprising the form of an unencapsulated crystal. For example, microencapsulation may at least partially prevent inhalation of unencapsulated crystals comprising epinephrine or a salt thereof. In some instances, unencapsulated crystals such as epinephrine crystals may cause irritation of the respiratory tract of a subject during inhalation. The irritation may be caused by crystal geometry and structure. For example, a crystal may have sharp angles and edges that may cause irritation, damage or both of the respiratory tract during inhalation. In some instances, crystal geometry and structure may be controlled by the spray drying process. Microencapsulation may generate crystals with amorphous structure. In some instances, an amorphous crystal may lack sharp edges and angles. In some cases, an amorphous crystal may have a rounded edge. In some instances, an amorphous crystal may have increased bioavailability.

Epinephrine, or a salt thereof, or an insulin may be encapsulated to provide a longer shelf life. In some cases, an active ingredient can be encapsulated with a diluent. The diluents may be aqueous, or solvent based and use animal or plant materials. In some cases, the diluent may comprise alcohols: e.g., ethanol, butanol, 2-ethylhexanol, isobutanol, isopropanol, methanol, propanol, propylene glycol; ketones: e.g., acetone, methyl ethyl ketone, methyl isobutyl ketone, methyl isopropyl ketone, mesityl oxide, trichloroethylene; halogenated solvents: e.g., ethylene bromide, chloroform, ethylene chloride, dichloromethane, tetrachloroethylene, carbon tetrachloride; amides: e.g., dimethylformamide; ethers: e.g., 1,4-dioxane, butyl ether, ethyl ether, di-isopropyl ether, tetrahydrofuran, tert-butyl methyl ether; sulfur containing solvent: e.g., dimethyl sulfoxide; amines: e.g., pyridine; nitriles: e.g., acetonitrile; esters: e.g., ethyl acetate; aliphatic hydrocarbons: e.g., cyclohexane hexane; aromatic hydrocarbons: e.g., toluene xylene; water or any combinations thereof. In some cases, the diluent may comprise benzene, carbon tetrachloride, 1,2-dichloroethane, 1,1-dichloroethene, 1,1,1-trichloroethane, acetonitrile, chlorobenzene, chloroform, cyclohexane, 1,2-dichloroethene, dichloromethane, 1,2-dimethoxyethane, N,N-dimethylacetamide, N,N-dimethylformamide, 1,4-dioxane, 2-ethoxyethanol, ethylene glycol, formamide, hexane, methanol, 2-methoxyethanol, methylbutylketone, methylcyclohexane, n-methylpyrrolidone, nitromethane, pyridine, sulfolane, tetralin, toluene, 1,1,2-trichloroethylene, xylene or any combinations thereof.

The core active ingredient may be microencapsulated with an amphipathic molecule that has both a polar end (“hydrophilic”) and non-polar end (“hydrophobic”). In some cases, a hydrophilic end of an amphipathic molecule may interact with core material. In some cases, a hydrophobic end of an amphipathic molecule may interact with core material. This hydrophilic and hydrophobic structure may enable the molecule to microencapsulate an active ingredient and form a microsphere. In some instances, the microencapsulated particle may have a hydrophilic exterior and a hydrophobic interior. In some instances, the microencapsulated particle may have a hydrophobic exterior and a hydrophilic interior. The microencapsulation process may coat the active ingredient, which is the core, by the amphipathic encapsulating agent, which is the wall material, so that the active ingredient is at least partially surrounded by a wall of the amphipathic material. For example, hydroxypropyl methylcellulose acetate succinate (HPMCAS) may be an amphipathic molecule used to coat epinephrine or a salt thereof. The microencapsulation blend may be a spray dried dispersion, that may be fed into a spray dry system to create a hard-outer coating on the microcapsules.

In some embodiments, a particle that has previously been microencapsulated can be microencapsulated with another coating. For example, an initial coating can be applied to cover the fill (e.g., the active ingredient) then a second coating can be applied to the first coating. In some cases, a third coating can be applied to the second coating. In some cases, a fourth coating can be applied to the third coating. In some cases, a fifth coating can be applied to a fourth coating. In some cases, a sixth coating can be applied to the fifth coating. In some cases, a seventh coating can be applied to a sixth coating. In some cases, an eighth coating can be applied to the seventh coating. In some cases, a ninth coating can be applied to the eighth coating. In some cases, additional coatings can be applied to the ninth coating.

In some embodiments, after each microencapsulation process, the suspension can be spray dried to create a dry powder product. In some embodiments, after a particle has been microencapsulated, an addition coating, for example, an enteric coating, or any coating described herein may be applied by a fluidized bed system.

The wall material may form a film that is cohesive with the core active ingredient. A wide variety of coating materials are available for encapsulation, e.g., traditional coating materials like inert polymers and pH sensitive ones as carboxylate and amino derivatives, which swell or dissolve according to the degree of cross-linking; some innovative coating polymers have also been developed for applications particularly among the bioadhesives and mucoadhesives. In some cases, the coating material may be hydrophilic polymers, hydrophobic polymers or a combination of both. In some cases, a microcapsule shell may comprise an amphipathic molecule. In some cases, the coating material may be a gelatin, a polyvinyl alcohol, an ethyl cellulose, a cellulose acetate phthalate or a styrene maleic anhydride. In some instances, the coating material may not react with the pharmaceutical ingredient. In some cases, a microcapsule shell may comprise a hydroxypropyl methylcellulose (“HPMC”), a hydroxypropyl methylcellulose acetate succinate (“HPMCAS”), an hydroxypropyl cellulose (“HPC”), a cyclodextrin, a maltodextrin, a povidone, a copovidone and others. In some instances, a microcapsule shell may comprise HPMCAS-LG, HPMCAS-MG, HPMCAS-HG or HPMC-P or a combination thereof. In some instances, a microcapsule shell may comprise a different grade of HPMC or HPMCAS. For example, a microcapsule shell may comprise an E5, an E50, or a K4M grade of HPMC. In another example, a microcapsule shell may comprise a L, a M, or an H grade of HPMCAS. In some cases, a microcapsule shell may comprise a HPMCAS. In some cases, a microcapsule shell can comprise fumaryl diketopiperazine (FDKP), polysorbate 80, both. In some cases, a microcapsule shell can comprise a trehalose, a lipids, a milk sugar. mannitol (e.g., pearlitol 200), a sugar, or any combination thereof. In some cases, a microcapsule shell may comprise a gelatin, a cornstarch, a polyvinylpyrrolidone (PVP), an oligosaccharide, a long chain sugar or any combination thereof. In some cases, a microcapsule shell may comprise a fatty acid, a liposome, an amino acid, a natural oil and a sugar, a trehalose, a dextran, a natural oil, a synthetic oil or a combination thereof. In some instances, an amino acid may comprise a glutamic acid, an aspartic acid, a lysine, a tryptophan, a tyrosine, a methionine or a combination thereof. In some cases, a fatty acid may comprise a polyunsaturated fatty acid, an essential fatty acid, a conjugated fatty acid, a short chain fatty acid, a medium chain fatty acid, a long chain fatty acid, a very long chain fatty acid, a saturated fatty acid, an unsaturated fatty acid, a monounsaturated fat, or any combination thereof. In some cases, a fatty acid may comprise an omega-3, an omega-5 fatty acid, an omega-6, an omega-7 fatty acid, an omega-9 fatty acid, an omega-10 fatty acid, an omega-11 fatty acid, an omega-12 fatty acid, or a combination thereof. In some cases, a natural oil may comprise soybean oil, a vegetable oil, a food oil, evening primrose oil, borage oil, blackcurrant seed oil, flax or linseed oil, rapeseed or canola oil, corn oil, almond oil, avocado oil, brazil nut oil, canola oil, cashew oil, chia seed oil, cocoa butter oil, coconut oil, corn oil, cottonseed oil, flaxseed/linseed oil, grape seed oil, hemp seed oil, vigna mungo oil, mustard oil, olive oil, palm oil, peanut oil, pecan oil, perilla oil, rice bran oil, safflower oil, sesame oil, soybean oil, walnut oil, sunflower oil, cottonseed oil, palm oil, or a combination thereof. In some cases, a microcapsule shell may increase or decrease active ingredient release kinetics. In some cases, a microcapsule shell may increase or decrease bioavailability. In some cases, microencapsulation of epinephrine, or a salt thereof, may produce about: 5% to about 70%, 5% to about 10%, 5% to about 20%, 10% to about 30%, 15% to about 40%, 25% to about 40%, 10% to about 60%, or about 20% to about 50% more bioavailability of the epinephrine or the salt thereof as compared to a epinephrine or a salt thereof that is not encapsulated when inhaled by a subject. In some cases, microencapsulation of an insulin may produce about: 5% to about 70%, 5% to about 10%, 5% to about 20%, 10% to about 30%, 15% to about 40%, 25% to about 40%, 10% to about 60%, or about 20% to about 50% more bioavailability of the insulin as compared to an insulin that is not encapsulated when inhaled by a subject. The wall material may be biodegradable and biocompatible with the pharmaceutical ingredient. In some cases, a microcapsule may be produced by dissolving or mixing the pharmaceutical ingredient in a solvent containing the shell material to produce a liquid suspension. For example, HPMCAS may be dissolved with ethanol and water and a pharmaceutical compound may be added the liquid suspension. In some instances, the pharmaceutical compound may not dissolve in the liquid suspension. In some instances, the pharmaceutical compound may dissolve in the liquid suspension. The liquid suspension may be dried with a spray drying technique described herein or by another method.

In some cases, the average wall thickness may of a microencapsulated particle may be about: 500 nm, 550 nm, 600 nm, 650 nm, 700 nm, 750 nm, 800 nm, 850 nm, 900 nm, 950 nm, 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, or 30 μm. In some cases, the wall thickness may of a microencapsulated particle may range from about: 1 μm to about 10 μm, 1 μm to about 5 μm, 2 μm to about 7 μm, 3 μm to about 8 μm, 5 μm to about 10 μm, 5 μm to about 15 μm, or 1 μm to about 30 μm. In some instances, the wall thickness of a microencapsulated particle may increase by increasing the ratio of the wall material to the core material prior to spray drying. In some cases, the ratio of wall material to core material (weight/weight) may be about: 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 60:1, 70:1, 80:1, 90:1, or 100:1. In some cases, the ratio of the wall material to core material (weight/weight) may be about 10:1.

In some aspects, in a plurality of microencapsulated particles about: 1%, 5%, 10%, 15%, 20%, 25%, 30%, 35%, 40%, 45%, 50%, 55%, 60%, 65%, 70%, 75%. 80%, 90%, 95%, 99% or 100% of the microencapsulated particles may comprise a core substantially encapsulated by a wall material. In some cases, in a plurality of microencapsulated particles about: 1% to about 50%, 1% to about 20%, 1% to about 10%, 5% to about 25%, 10% to about 40%, 10% to about 60%, 20% to about 70%, 20% to about 50%, 30% to about 80%, 40% to about 90%, 50% to about 75%, 60% to about 80%, 70% to about 90%, 75% to about 95%, 80% to about 90%, 80% to about 99%, 85% to about 100%, or 90% to about 100% of the microencapsulated particles may comprise a core substantially encapsulated by a wall material. In some cases, in a plurality of microencapsulated particles not all of the core material may be encapsulated by the wall material.

In some aspects, microencapsulated particles have a mean, a median, or a mode particle diameter of less than about: 500 nm, 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm or 20 μm. In some aspects, microencapsulated particles have a mean, a median, or a mode particle diameter of more than about: 500 nm, 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm or 20 μm. In some aspects, microencapsulated particles have a mean, a median, or a mode particle diameter ranging from about: 500 nm to about 5 μm, 1 μm to about 10 μm, 1 μm to about 5 μm, 2 μm to about 7 μm, 3 μm to about 8 μm, 5 μm to about 10 μm, 1 μm to about 20 μm, or 5 μm to about 15 μm.

The core material may be the material over which coating has to be applied to serve the specific purpose. Core material may be in form of solids or droplets of liquids and dispersions. In some cases, core material may comprise epinephrine or a salt thereof. In some cases, core material may comprise another sympathomimetic drug or a salt thereof. In some cases, core material may comprise an insulin. The composition of core material may vary and thus furnish definite flexibility and allow effectual design and development of the desired microcapsule properties. A substance may be microencapsulated for a number of reasons. Examples may include protection of reactive material from their environment, safe and convenient handling of the materials which may be otherwise toxic or noxious, taste masking, means for controlled or modified release properties means of handling liquids as solids, preparation of free flow powders and in modification of physical properties of the drug. For example, encapsulation may improve solubility and dissolution and therefore increase bioavailability of an active ingredient such as epinephrine or a salt thereof. Microencapsulation may be used to increase the stability, improve the handling properties of compounds, facilitate higher bioavailability when reconstituted or administered, or any combination thereof. For example, microencapsulated epinephrine or microencapsulated insulin may not require refrigeration and may not lose efficacy when exposed to a high temperature for a period of time. In some cases, the shelf life of encapsulated epinephrine or encapsulated insulin may be extended from about: 4, 5, 6, 7, 8, 9, 10, 11, 12, 13, 14, 15, 16, 17, 18, 19, 20, 21, 22, 23, 24 or more months as compared to a liquid formation comprising epinephrine. In some instances, inhalable epinephrine and/or insulin doesn't require a needle for administration which may eliminate the potential for a needle breaking during an emergency.

In some instances, the core diameter of a microencapsulated particle may be about: 100 nm (nanometer), 150 nm, 200 nm, 250 nm, 300 nm, 350 nm, 400 nm, 450 nm, 500 nm, 550 nm, 600 nm, 650 nm, 700 nm, 750 nm, 800 nm, 850 nm, 900 nm, 950 nm, 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, or 30 μm. In some cases, the core diameter of a microencapsulated particle may range from about: 100 nm to about 250 nm, 100 nm to about 500 nm, 100 nm to about 1 μm, 500 nm to about 1 μm, 1 μm to about 10 μm, 1 μm to about 5 μm, 2 μm to about 7 μm, 3 μm to about 8 μm, 5 μm to about 10 μm, 5 μm to about 15 μm, or 1 μm to about 30 μm. In some instances, the core may comprise about: 1%, 5%, 10%, 15%, 20%, 25%, 30%, 35%, 40%, 45%, 50%, 55%, 60%, 65%, 70%, 75%. 80%, 90%, 95% or 99% of the total microcapsule content (e.g., total weight of the core and wall material). In some instances, the core may comprise about: 1% to about 50%, 1% to about 20%, 1% to about 10%, 5% to about 25%, 10% to about 40%, 10% to about 60%, 20% to about 70%, 20% to about 50%, 30% to about 80%, 40% to about 90%, 50% to about 75%, or 1% to about 99% of the total microcapsule content.

In some aspects, a method of microencapsulation may comprise at least partially dissolving the coating material (e.g., HPMC, HPMCAS, HPC) in a solvent such as ethanol, water, a mixture thereof, or another solvent. In some cases, particles of epinephrine (or a liquid form) or a salt thereof may be added to the solution of the coating material and the solvent to create a suspension/mixture of the epinephrine and the coating material dissolved in the solvent. In some cases, particles of an insulin (or a liquid form of an insulin) may be added to the solution of the coating material and the solvent to create a suspension/mixture of the insulin and the coating material dissolved in the solvent. In some instances, the epinephrine may not dissolve in the suspension and may remain in suspension. In some instances, the insulin may not dissolve in the suspension and may remain in suspension. In some instances, the epinephrine or the insulin may dissolve in the suspension. The suspension may be mixed to an at least partially uniform mixture and spray dried. The coating may at least partially encapsulate the epinephrine or salt thereof or an insulin. In some cases, the epinephrine or salt thereof may be amorphous epinephrine or a salt thereof. In some cases, the encapsulation of the epinephrine or the insulin may be a spherical, round, oval, or any shape structure.

### Methods of Making

Also disclosed herein are methods of making the powdery pharmaceutical composition to select sizes.

In some aspects, a method of making the powdery pharmaceutical composition may comprise mixing particles of a first pharmaceutically acceptable excipient and at least one of: particles of active pharmaceutical ingredients or salts, particles of active pharmaceutical ingredients or salts at least partially contained within a second pharmaceutically acceptable excipient, particles of active pharmaceutical ingredients or salts at least partially contained within a pore of a second pharmaceutically acceptable excipient, particles of active pharmaceutical ingredients or salts encapsulated in a coating material, or any combination thereof. In some aspects, a method of making the powdery pharmaceutical composition may comprise mixing particles in a mixer. In some instances, “a first pharmaceutically acceptable excipient” as used herein may comprise a pharmaceutically acceptable excipient.

In some aspects, the method of making the powdery pharmaceutical composition may comprise mixing the particles described herein. In some instances, at least a portion of the particles of a pharmaceutically acceptable excipient may have a particle diameter ranging from about 50 micrometers to about 200 micrometers, as measured by a particle size analyzer using laser diffraction. In some cases, at least a portion of the particles comprising encapsulated epinephrine or encapsulated insulin may have a particle diameter ranging from about 500 nanometers to about 15 micrometers, or about 1 micrometer to about 20 micrometers, as measured by a particle size analyzer using laser diffraction. In some cases, in a human clinical trial, the powdery pharmaceutical composition, when inhaled into the lungs, may provide in at least part of the humans in the clinical trial a time to peak plasma concentration (Tmax) of the active ingredient or the salt thereof ranging from about 1 minute to about one hour, or from about 1 minute to about ten minutes.

In some aspects, at least a portion of the particles of a pharmaceutically acceptable excipient may have a particle diameter ranging from about: 30 μm (micrometers) to about 60 μm, 50 μm, to about 200 μm, 60 μm to about 80 μm, 70 μm to about 100 μm. 90 μm to about 130 μm, 110 μm to about 150 μm, 130 μm to about 180 μm, 150 μm to about 200 μm, 190 μm to about 250 μm, or 200 μm to about 400 μm. In some cases, particles of a pharmaceutically acceptable excipient may have a particle diameter of more than about: 30 μm, 40 μm, 45 μm, 50 μm, 55 μm, 60 μm, 65 μm, 70 μm, 75 μm, 80 μm, 85 μm, 90 μm, 95 μm, 100 μm, 105 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, or 400 μm. In some cases, particles of a pharmaceutically acceptable excipient may have a particle diameter of less than about: 30 μm, 40 μm, 45 μm, 50 μm, 55 μm, 60 μm, 65 μm, 70 μm, 75 μm, 80 μm, 85 μm, 90 μm, 95 μm, 100 μm, 105 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, or 400 μm. In some cases, the particles of a pharmaceutically acceptable excipient may range from about 50 μm to about 100 μm, which may be preferred when inhaled or administered intranasally for deposit on the oropharynx. In some instances, particle size as may comprise the diameter, the radius, or length of a particle. In some instances, particle size may be a measure of the mean, the median or the mode of a plurality of particles.

In some aspects, i) particles of an active ingredient or a pharmaceutically acceptable salt thereof or ii) at least partially encapsulated particles comprising an active ingredient or a pharmaceutically acceptable salt thereof may have particle diameters ranging from about: 100 nm (nanometer) to about 500 nm, 300 nm to about 800 nm, 700 nm to about 1.2 μm, 1 μm to about 3 μm, 2 μm to about 4 μm, 3 μm to about 6 μm, 5 μm to about 8 μm, 6 μm to about 9 μm, 7 μm to about 10 μm, 8 μm to about 11 μm, 9 μm to about 13 μm, 10 μm to about 15 μm, 12 μm to about 20 μm, 14 μm to about 25 μm, or 18 μm to about 30 μm. In some cases, i) particles of an active ingredient or a pharmaceutically acceptable salt thereof or ii) at least partially encapsulated particles comprising an active ingredient or a pharmaceutically acceptable salt thereof may have a particle diameter of less than about: 30 nm, 50 nm, 60 nm, 70 nm, 80 nm, 90 nm, 100 nm, 150 nm, 200 nm, 250 nm, 300 nm, 350 nm, 400 nm, 450 nm, 500 nm, 550 nm, 600 nm, 650 nm, 700 nm, 750 nm, 800 nm, 850 nm, 900 nm, 950 nm, 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, or 30 μm. In some cases i) particles of an active ingredient or a pharmaceutically acceptable salt thereof or ii) at least partially encapsulated particles comprising an active ingredient or a pharmaceutically acceptable salt thereof may have a particle diameter of more than about: 30 nm, 50 nm, 60 nm, 70 nm, 80 nm, 90 nm, 100 nm, 150 nm, 200 nm, 250 nm, 300 nm, 350 nm, 400 nm, 450 nm, 500 nm, 550 nm, 600 nm, 650 nm, 700 nm, 750 nm, 800 nm, 850 nm, 900 nm, 950 nm, 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, or 30 μm. In some cases, i) particles of an active ingredient or a pharmaceutically acceptable salt thereof or ii) at least partially encapsulated particles comprising an active ingredient or a pharmaceutically acceptable salt thereof may be in about 1 μm to about 5 μm, which may be preferred when inhaled or administered intranasally for absorption into lung alveoli.

In some aspects, a particles or compositions described herein may have a tap density of more than about: 0.1 grams/centimeter (g/cm³), 0.2 g/cm³, 0.3 g/cm³, 0.4 g/cm³, 0.5 g/cm³, 0.6 g/cm³, 0.7 g/cm³, 0.8 g/cm³, 0.9 g/cm³, 1.0 g/cm³, 1.1 g/cm³, or 1.2 g/cm³. In some aspects, a particles described herein may have a tap density of less than about: 0.1 g/cm³, 0.2 g/cm³, 0.3 g/cm³, 0.4 g/cm³, 0.5 g/cm³, 0.6 g/cm³, 0.7 g/cm³, 0.8 g/cm³, 0.9 g/cm³, 1.0 g/cm³, 1.1 g/cm³, or 1.2 g/cm³. In some cases, particles or compositions described herein may have a tap density of more than about 0.6 g/cm³ or 0.7 g/cm³. In some cases, particles or compositions described herein may have a tap density of about 0.6 g/cm³ or 0.7 g/cm³. In some cases, tap density of a powder may be the ratio of the mass of the powder to the volume occupied by the powder after it has been tapped for a defined period of time. In some cases, tap density may be a measure of the envelope mass density characterizing a particle. The envelope mass density of a particle of a statistically isotropic shape may be defined as the mass of the particle divided by the minimum sphere envelope volume within which it may be enclosed. Features which may contribute to low tap density include irregular surface texture, porous structure or a combination thereof. Tap density may be measured by using instruments known to those skilled in the art such as the Dual Platform Microprocessor Controlled Tap Density Tester (Vankel, N.C.) or a GeoPyc™ instrument (Micrometrics Instrument Corp., Norcross, Ga.).

In some aspects, particles of an active ingredient or a pharmaceutically acceptable salt thereof may be mixed in sizes. In some cases, the mixed sizes may change the release time of the drug. For example, particles with small sizes (e.g., about 1 μm to about 5 μm) may be readily absorbed into the blood stream while particles larger than about 10 μm may take longer to be absorbed into the blood stream. In some cases, particles with diameters of about 1 μm to about 10 μm may be inhaled into the lung while larger particles may be deposited onto the oropharynx. In some cases, particles with diameters of about 1 μm to about 5 μm may absorb faster than particles with diameters of about 7 μm to about 10 μm. In some aspects, the particles with sizes of about 7 μm to about 10 μm may be mixed with particles with sizes of about 1 μm to about 5 μm. In some aspects, the weight to weight ratio of the particles with diameters of about 7 μm to about 10 μm to the particles with sizes of about 1 μm to about 5 μm may range from about 1:1 to about 1:2, about 1:1 to about 1:3, about 1:1 to about 1:4, about 1:1 to about 1:5, about 1:1 to about 1:8, about 1:1 to about 1:10, about 1:2 to about 1:3, about 1:2 to about 1:4, about 1:2 to about 1:5, about 1:2 to about 1:8, about 1:2 to about 1:10, about 1:3 to about 1:4, about 1:3 to about 1:5, about 1:3 to about 1:8, about 1:3 to about 1:10, about 1:4 to about 1:5, about 1:4 to about 1:8, about 1:4 to about 1:10, about 1:5 to about 1:8, about 1:5 to about 1:10, or 1:8 to about 1:10. In some aspects, the weight to weight ratio of the particles with diameters of about 1 μm to about 5 μm to the particles with sizes of about 7 μm to about 10 μm may range from about 1:1 to about 1:2, about 1:1 to about 1:3, about 1:1 to about 1:4, about 1:1 to about 1:5, about 1:1 to about 1:8, about 1:1 to about 1:10, about 1:2 to about 1:3, about 1:2 to about 1:4, about 1:2 to about 1:5, about 1:2 to about 1:8, about 1:2 to about 1:10, about 1:3 to about 1:4, about 1:3 to about 1:5, about 1:3 to about 1:8, about 1:3 to about 1:10, about 1:4 to about 1:5, about 1:4 to about 1:8, about 1:4 to about 1:10, about 1:5 to about 1:8, about 1:5 to about 1:10, or 1:8 to about 1:10. In some aspects, the particles with larger sizes (about m to about 20 μm) may be mixed with particles with smaller sizes (about 1 μm to about m). In some aspects, the weight to weight ratio of the particles with larger sizes (about 10 m to about 20 μm) to the particles with smaller sizes (about 1 μm to about 10 μm) may range from about 1:1 to about 1:2, about 1:1 to about 1:3, about 1:1 to about 1:4, about 1:1 to about 1:5, about 1:1 to about 1:8, about 1:1 to about 1:10, about 1:2 to about 1:3, about 1:2 to about 1:4, about 1:2 to about 1:5, about 1:2 to about 1:8, about 1:2 to about 1:10, about 1:3 to about 1:4, about 1:3 to about 1:5, about 1:3 to about 1:8, about 1:3 to about 1:10, about 1:4 to about 1:5, about 1:4 to about 1:8, about 1:4 to about 1:10, about 1:5 to about 1:8, about 1:5 to about 1:10, or 1:8 to about 1:10.

In some aspects, active ingredient particles may be produced by spray drying. In some cases, encapsulated active ingredient particles may be produce by spray drying. In some instances, active ingredient particles may be produced by another method. In some instances, active ingredient particles may be produced by air-jet micronization, spiral milling, controlled precipitation, high-pressure homogenization, or cryo-milling.

In some aspects, particles that are not of the first pharmaceutically acceptable excipient, may have particle diameters ranging from about 1 μm to about 20 μm. In some aspects, particle diameters may be measured by a particle analyzer using laser diffraction (LD), static light scattering, dynamic light scattering (DLS), or nanoparticle tracking analysis (NTA).

In some aspects, active ingredient particles may comprise a sympathomimetic drug such as epinephrine or a salt of epinephrine. In some instances, epinephrine may be blended with an excipient such as lactose or a salt thereof. In some instances, an insulin may be blended with an excipient such as a lactose or a salt thereof. In some instances, an excipient may comprise lactose, microcrystalline cellulose, cellulose, mannitol, sorbitol, starch, starch glycolate, hydroxypropyl methylcellulose, hydroxypropyl methylcellulose acetate succinate, a cyclodextrin, maltodextrin, croscarmellose sodium, corn starch, carrageenan, sorbitol, maltitol, glucose, a pharmaceutically acceptable salt of any of these, or any combination thereof. In some instances, the epinephrine, or a salt thereof and an excipient may be used to treat COVID-19. For example, epinephrine or a salt thereof may be used to treat a cytokine storm from COVID-19. In some cases, epinephrine or a salt thereof may be used to treat an allergic reaction to a vaccine, for example a COVID-19 vaccine. In some cases, encapsulated or unencapsulated epinephrine, or a salt thereof may have a mean particle diameter of about 30 nm, 50 nm, 60 nm, 70 nm, 80 nm, 90 nm, 100 nm, 150 nm, 200 nm, 250 nm, 300 nm, 350 nm, 400 nm, 450 nm, 500 nm, 550 nm, 600 nm, 650 nm, 700 nm, 750 nm, 800 nm, 850 nm, 900 nm, 950 nm, 1 m, 2 μm, 3 μm, 4 μm, 5 μm, 6 μm, 7 μm, 8 μm, 9 μm, 10 μm, 11 μm, 12 μm, 13 μm, 14 μm, m, 16 μm, 17 μm, 18 μm, 19 μm, or 20 μm. In some cases, encapsulated or unencapsulated insulin may have a mean particle diameter of about 30 nm, 50 nm, 60 nm, 70 nm, 80 nm, 90 nm, 100 nm, 150 nm, 200 nm, 250 nm, 300 nm, 350 nm, 400 nm, 450 nm, 500 nm, 550 nm, 600 nm, 650 nm, 700 nm, 750 nm, 800 nm, 850 nm, 900 nm, 950 nm, 1 μm, 2 μm, 3 μm, 4 μm, m, 6 μm, 7 μm, 8 μm, 9 μm, 10 μm, 11 μm, 12 μm, 13 μm, 14 μm, 15 μm, 16 μm, 17 μm, 18 μm, 19 μm, or 20 μm. In some cases, an excipient or a salt thereof may have a mean particle diameter of about: 30 μm, 40 μm, 45 μm, 50 μm, 55 μm, 60 μm, 65 μm, 70 μm, 75 μm, 80 μm, 85 μm, 90 μm, 95 μm, 100 μm, 105 μm, 110 μm, 115 μm, 120 μm, 125 μm, 130 μm, 135 μm 140 μm, 145 μm 150 μm, 155 μm 160 μm, 165 μm 170 μm, 175 μm 180 μm, 185 μm 190 μm, 195 μm, 200 μm, 205 μm, 210 μm, 215 μm 220 μm, 225 μm 230 μm, 235 μm 240 μm, 245 μm, or 250 μm. In some cases, epinephrine, or a salt thereof, or an insulin may be microencapsulated by a process described herein. In some instances, the shell of the microencapsulation comprises HPMCAS or HPMC. In some cases, microencapsulation of epinephrine, or the salt thereof, or an insulin by HPMCAS may provide faster absorption in the lungs. For example, microencapsulation with HPMCAS of epinephrine may provide increased absorption into the blood stream from the lungs. In some instances, microencapsulation may increase the solubility of an active ingredient. In some cases, microencapsulated epinephrine, or a salt thereof, or an insulin may be absorbed about: 10% to about 70%, 5% to about 10%, 5% to about 20%, 10% to about 30%, 15% to about 40%, 25% to about 40%, 35% to about 50%, 10% to about 60%, 40% to about 90%, or 20% to about 50% faster than epinephrine, or a salt thereof, or an insulin that is not microencapsulated. In some cases, microencapsulated epinephrine, or a salt thereof, or an insulin may be absorbed after inhalation into the blood stream in about: 5 seconds to about 30 seconds, 5 seconds to about 20 seconds, 10 seconds to about 20 seconds, 10 seconds to about 30 seconds, 10 seconds to about 60 seconds, 20 seconds to about 40 seconds, 30 second to about 60 seconds, 30 seconds to about 2 minutes, or 1 minute to about 2 minutes. In some instances, a epinephrine, or a salt thereof, or an insulin may be mixed with an excipient prior to adding to a capsule. In some cases, the mixing may comprise blending in a blender such as a V-type blender. In some cases, epinephrine, or a salt thereof, or an insulin may be mixed in a V-type blender with an excipient. A V-type blender may include a Patterson Kelly/PK Blender, a Gemco or a Ross blender. In some instances, blending may be high shear or low shear blending. In some cases, blending may be high speed or low speed blending. In some cases, the blending may distribute the active ingredient and the excipient evenly. In some cases, the weight to weight ratio of an epinephrine, or a salt thereof, and an excipient may be about: 1:1 (epinephrine to excipient), 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 55:1, 60:1, 65:1, 70:1, 75:1, 80:1, 85:1, 90:1, 95:1, or 100:1. In some cases, the weight to weight ratio of an epinephrine, or a salt thereof, and an excipient may be about: 1:1 (epinephrine to excipient), 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, 1:55, 1:60, 1:65, 1:70, 1:75, 1:80, 1:85, 1:90, 1:95, or 1:100. In some aspects, an active ingredient or a pharmaceutically acceptable salt thereof (e.g., epinephrine or the pharmaceutically acceptable salt thereof) may comprise at least about:0.01%, 0.1%, 1%, 2%, 3%, 4%, 5%, 6%, 7% 8%, 9% or 10% by weight of a pharmaceutical composition. The blending may not cause the excipient particle to be coated by the epinephrine particle or the salt thereof. In some instances, the epinephrine, or a salt thereof, and an excipient may be administered via inhalation by a dry powder inhaler. In some cases, a dry powder inhaler does not comprise a propellent. In some cases, a dry powder inhaler does not comprise a chlorofluorocarbon, a hydrofluorocarbon, a fluorocarbon or any combination thereof as a propellent. In some cases, a dry powder inhaler is not pressurized. In some instances, inhalation administration of epinephrine, or a salt thereof, and an excipient may produce about: 90%, 91%, 92%, 93%, 94%, 95%, 96%, 97%, 98%, 99% or 100% bioavailability of the epinephrine, or a salt thereof. In some cases, the Tmax (e.g., the time required to reach the maximum concentration of a drug in the plasma) may be about: 5 min, 10 min, 15 min, 20 min, 25 min, 30 min, 35 min, 40 min, 45 min, 50 min, 55 min, 60 min, 65 min, 70 min, 75 min, 80 min, 85 min, 90 min, 95 min, 100 min, 105 min, 110 min, 115 min, 120 min, 130 min, 140 min, 150 min, 160 min, 170 min, 180 min, 190 min, 200 min, 210 min, 220 min, 230 min, 240 min, 250 min, 260 min, 270 min, 280 min, 290 min, or 300 min for epinephrine, or a salt thereof. In some cases, a composition comprising epinephrine, or a salt thereof, may comprise epinephrine, or a salt thereof in an amount of about: 10 μg, 25 μg, 50 μg, 75 μg, 100 μg, 150 μg, 200 μg, 220 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, or 25 mg.

In some cases, the weight to weight ratio of an insulin, or a derivative thereof and the excipient may be about: 1:1 (insulin to excipient), 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 55:1, 60:1, 65:1, 70:1, 75:1, 80:1, 85:1, 90:1, 95:1, or 100:1. In some cases, the weight to weight ratio of an insulin, or a derivative thereof and the excipient may be about: 1:1 (insulin to excipient), 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, 1:55, 1:60, 1:65, 1:70, 1:75, 1:80, 1:85, 1:90, 1:95, 1:100, 1:1000 or 1:10000. In some aspects, an active ingredient or a pharmaceutically acceptable salt thereof (e.g., an insulin or a derivative thereof) may comprise at least about: 0.01%, 0.1%, 1%, 2%, 3%, 4%, 5%, 6%, 7%, 8%, 9%, or 10% by weight of a pharmaceutical composition. In some cases, the blending may not cause the excipient particle to be coated by the insulin particle. In some instances, the insulin, or the derivative thereof, and an excipient may be administered via inhalation by a dry powder inhaler. In some cases, a dry powder inhaler does not comprise a propellent. In some cases, a dry powder inhaler does not comprise a chlorofluorocarbon, a hydrofluorocarbon, a fluorocarbon or any combination thereof as a propellent. In some cases, a dry powder inhaler is not pressurized. In some instances, inhalation administration of an insulin or a derivative thereof, and an excipient may produce about: 90%, 91%, 92%, 93%, 94%, 95%, 96%, 97%, 98%, 99% or 100% bioavailability of the insulin, or the derivative thereof. In some cases, the Tmax (e.g., the time required to reach the maximum concentration of a drug in the plasma) may be about: 5 min, 10 min, 15 min, 20 min, 25 min, 30 min, 35 min, 40 min, 45 min, 50 min, 55 min, 60 min, 65 min, 70 min, 75 min, 80 min, 85 min, 90 min, 95 min, 100 min, 105 min, 110 min, 115 min, 120 min, 130 min, 140 min, 150 min, 160 min, 170 min, 180 min, 190 min, 200 min, 210 min, 220 min, 230 min, 240 min, 250 min, 260 min, 270 min, 280 min, 290 min, or 300 min for an insulin, or a derivative thereof. In some cases, a composition comprising an insulin, or a derivative thereof, may comprise an insulin, or a derivative thereof in an amount of about: 1 μg, 2 μg, 3 μg, 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 g, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 150 μg, 200 μg, 220 μg, 250 μg, 300 μg, 350 μg, 400 μg, 450 μg, 500 kg, 550 μg, 600 μg, 650 μg, 700 μg, 750 μg, 800 μg, 850 μg, 900 μg, 950 μg, 1000 μg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, or 25 mg.

In some aspects, a method of making a powdery pharmaceutical composition, may comprise blending: i) particles of a pharmaceutically acceptable excipient; and ii) a plurality of spray dried particles, each particle of the plurality of spray dried particles may comprise a sympathomimetic agent (e.g., epinephrine) or a pharmaceutically acceptable salt thereof, substantially encapsulated in a coating material. In some cases, each particle of the plurality of spray dried particles may comprise an insulin. In some cases, a portion of the plurality of spray dried particles comprising the sympathomimetic agent, or the pharmaceutically acceptable salt thereof substantially encapsulated in the coating material may have a particle diameter ranging from about 1 micrometer to about 10 micrometers, or from about 1 micrometer to about 5 micrometers, as measured by a particle analyzer using laser diffraction. In some cases, a portion of the plurality of spray dried particles comprising the insulin substantially encapsulated in the coating material may have a particle diameter ranging from about 1 micrometer to about 10 micrometers, or from about 1 micrometer to about 5 micrometers, as measured by a particle analyzer using laser diffraction. In some instances, the coating material may comprise a hydroxypropyl methylcellulose (HPMC), a hydroxypropyl methylcellulose acetate succinate (HPMCAS), a cyclodextrin, a maltodextrin, a povidone, a copovidone or any combination thereof.

In some aspects, a powdery pharmaceutical composition may be produced by a process comprising: a) mixing the particles comprising a sympathomimetic agent (e.g., epinephrine) or a pharmaceutically acceptable salt thereof, a coating material, and a solvent and b) spray drying the mixed particles comprising the sympathomimetic agent or the pharmaceutically acceptable salt thereof, the coating material, and the solvent. In some cases, the spray dried particles may be mixed or blended with a pharmaceutically acceptable excipient to make a powdery pharmaceutical composition. In some cases, the mixing can comprise contacting.

In some aspects, a powdery pharmaceutical composition may be produced by a process comprising: a) mixing the particles comprising an insulin, a coating material, and a solvent and b) spray drying the mixed particles comprising the insulin, the coating material, and the solvent. In some cases, the spray dried particles may be mixed or blended with a pharmaceutically acceptable excipient to make a powdery pharmaceutical composition.

### Packaging of the Powdery Pharmaceutical Compositions

In some aspects, the pharmaceutical composition may be contained within a capsule, a tablet, a gel, a gummy, a spray, an ointment, a paste, a jelly, an oil, a tincture, a lotion, a cream, a balm, a food, a drink, a liquid, a syrup, or any combination thereof.

In some aspects, the capsule may comprise a single-piece capsule, two-piece capsule, transparent capsule, non-transparent capsule, opaque capsule, slow-release capsule, extended-release capsule, standard-release capsule, rapid-release capsule, quick-release capsule, hard-shell capsule, soft gel capsule, gel capsule, hard gelatin capsule, soft gelatin capsule, animal-based capsule, vegetarian capsule, polysaccharide capsule, cellulose capsule, mucopolysaccharide capsule, tapioca capsule, hydroxypropylmethyl cellulose (HPMC) capsule, pullulan capsule, enteric capsule, uncoated capsule, coated capsule, capsule comprising titanium dioxide, fatty acids, waxes, shellac, plastics, plasticizers, glycerin, sorbitol, plant fibers, additives, preservatives, colorants, or any combination thereof.

In some aspects, the capsule having different sizes according to pharmaceutical composition requirements. In some aspects, the capsule size is: 000, 00, 0, 1, 2, 3, or 4. In some aspects, the capsule size may be 000. In some aspects, the capsule size may be 00. In some aspects, the capsule size may be 0. In some aspects, the capsule size may be 1. In some aspects, the capsule size may be 2. In some aspects, the capsule size may be 3. In some aspects, the capsule size may be 4. In some aspects, the capsule capacity varies from about 0.21 ml to about 1.37 ml.

In some aspects, the powdery pharmaceutical composition described herein when stored in a sealed container placed in a room at 25° C. and a room atmosphere having about 50 percent relative humidity, retains at least about: 80%, 90%, 95%, 96%, 97%, 98%, or 99% of the active ingredient or the salt thereof after 6 months, as measured by HPLC.

In some aspects, the pharmaceutical composition may be contained within a capsule, wherein the capsule may be loaded with about 25% to about 75% (by volume) with the powdery pharmaceutical composition. In some cases, the capsule may be loaded with about: 30%, 31%, 32%, 33%, 34%, 35%, 36%, 37%, 38%, 39%, or 40% (by volume) with a pharmaceutical composition described herein. In some aspects, the capsule may be loaded with about 25% to about 30%, about 25% to about 40%, about 25% to about 50%, about 25% to about 60%, about 25% to about 65%, about 25% to about 70%, about 25% to about 75%, about 30% to about 40%, about 30% to about 50%, about 30% to about 60%, about 30% to about 65%, about 30% to about 70%, about 30% to about 75%, about 40% to about 50%, about 40% to about 60%, about 40% to about 65%, about 40% to about 70%, about 40% to about 75%, about 50% to about 60%, about 50% to about 65%, about 50% to about 70%, about 50% to about 75%, about 60% to about 65%, about 60% to about 70%, about 60% to about 75%, about 65% to about 70%, about 65% to about 75%, or 70% to about 75%, (by volume) with the powdery pharmaceutical composition.

In some aspects, the content of the capsule comprises less than about: 10%, 9%, 8%, 7%, 6%, 5%, 4%, 3%, 2%, or 1% water by weight. In some aspects, the content of the capsule comprises less than about 50%, about 40%, about 30%, about 25%, about 20%, about 10%, about 5%, or 1% water by weight.

In some aspects, the total content of all gases in the capsule may be less than about: 10%, 9%, 8%, 7%, 6%, 5%, 4%, 3%, 2%, or 1% water by weight. In some aspects, the total content of all gases in the capsule may be less than about 50%, about 40%, about 30%, about 25%, about 20%, about 10%, about 5%, or 1% water by weight.

In some aspects, the capsule further comprises, in the volume not occupied by the powdery pharmaceutical composition, an inert gas. In some aspects, the inert gas comprises an elemental gas, a compound gas, a noble gas, helium, neon, argon, krypton, xenon, oganesson, compounds of noble gas, purified argon, purified nitrogen, nitrogen, sulfur hexafluoride, or any combination thereof. In some aspects, the inert gas comprises nitrogen. In some cases, the inert gas within a capsule may comprise at least about: 75%, 80%, 85%, 90%, or 95% of the gas on a volume-to-volume basis.

In some aspects, the pharmaceutical composition may be contained within a capsule, wherein the capsule may be at least in part contained within a device which may be a drug delivery device, an inhalation drug delivery device, a diffuser, an inhaler, a metered dose inhaler, a dry powder inhaler, a soft mist inhaler, or any combination thereof. In some aspects, the device may be an inhaler. In some cases, a dry powder inhaler does not comprise a propellent. In some cases, a dry powder inhaler may not be pressurized. In some cases, an inhaler can comprise a propellent. In some cases, an inhaler can comprise a chlorofluorocarbon, a hydrofluorocarbon, a fluorocarbon or any combination thereof as a propellent In some instances, a dry powder inhaler comprises breathing or inhaling an active ingredient or composition into the lungs. In some instances, a dry powder inhaler may be breath-activated, wherein when a subject breathes in through an inhaler, the inhaler releases particles (e.g., an active ingredient, excipient or both) which travel throughout the respiratory system. In some cases, a capsule may contain an active ingredient which may be pierced to release the particles prior to inhalation through a dry powder inhaler. In some instances, particle size and aerodynamics may affect travel throughout the respiratory system.

In some aspects, the pharmaceutical composition may be contained within a capsule, wherein the capsule may be at least in part contained within the device. In some aspects, the pharmaceutical composition may be contained within a capsule, wherein the capsule may be at least in part contained within the device, and wherein the device contains a sharp surface configured to puncture or slice the capsule. In some aspects, the pharmaceutical composition may be contained within a capsule, wherein the capsule may be at least in part contained within the device, and wherein the device contains a sharp surface configured to puncture or slice the capsule, and wherein, prior to administrating, the device may be actuated such that the sharp surface punctures or slices the capsule.

In some aspects, the pharmaceutical composition may be contained within a capsule, wherein the capsule may be at least in part contained within an inhaler, and wherein the inhaler contains a sharp surface configured to puncture or slice the capsule. In some aspects, the pharmaceutical composition may be contained within a capsule, wherein the capsule may be at least in part contained within an inhaler, and wherein the inhaler contains a sharp surface configured to puncture or slice the capsule, and wherein, prior to administrating, the inhaler may be actuated such that the sharp surface punctures or slices the capsule. In some aspects, the inhaler unit may be re-used via a process comprising replacing a spent capsule with a new capsule containing the powdery pharmaceutical composition. In some aspects, a component of the inhaler unit configured to at least in part hold the capsule may be temporarily at least partially separable from the inhaler unit. In some aspects, the capsule may be at least partially visible via an at least partially transparent material present in the inhaler unit.

### Delivery of the Pharmaceutical Composition for Treatment of Diseases

In some aspects, the administration of the pharmaceutical composition or the second therapeutic may be administered orally, intra nasally, intra ocular, anally, by injection, intra venously, intra muscularly, subcutaneously, intra peritoneally, trans dermally, or any combination thereof.

In some aspects, the administration of the pharmaceutical composition may be by inhalation. In some aspects, inhalation may be oral inhalation, intra nasal administration, or any combination thereof. In some aspects, the powdery pharmaceutical composition may be inhaled into human lungs. In some cases, inhaled may be inhalation through the mouth, for example with a dry powdered inhaler. In some cases, at least a portion of the excipient may deposit on the oropharynx. In some aspects, the powdery pharmaceutical composition, when inhaled into the lungs, provides a time to peak plasma concentration (Tmax) of the active ingredient or the salt thereof. The time to peak plasma concentration (Tmax) of the active ingredient or the salt thereof may range from about 1 minute to about one hour. In some aspects, the time to peak plasma concentration (Tmax) of the active ingredient or the salt thereof may range from about 1 minute to about ten minutes.

In some aspects, administering may be by oral ingestion, topical application, or inhalation. In some aspects, administering may comprise oral ingestion and the oral ingestion may comprise oral ingestion of a food, a liquid, a gel, a capsule, or any combination thereof. In some aspects, administering may comprise topical application and the topical application may comprise topical application of a lotion, a tincture, a balm, a cream, an oil, a gel, a butter, a liquid, a spray, an ointment, a paste, a jelly, or any combination thereof. In some aspects, administering may comprise inhalation and the inhalation may comprise inhalation by a diffuser, an inhaler, a nebulizer, or any combination thereof. In some aspects, administering may comprise inhalation and the inhalation may comprise inhalation by a diffuser. In some aspects, administering may comprise inhalation and the inhalation may comprise inhalation by a nebulizer. In some aspects, administering may be performed at least about: 1 time per day, 2 times per day, 3 times per day, 4 times per day, 5 times per day, 6 times per day or more than 6 times per day. In some cases, administering may be performed daily, weekly, monthly, or as needed. In some aspects, administering may be conducted one, twice, three, or four times per day. In some cases, administration may be provided by a subject (e.g., the patient), a health care provider, or both.

In some aspects, administering may be performed for about: 1 day to about 8 days, 1 week to about 5 weeks, 1 month to about 12 months, 1 year to about 3 years, 3 years to about 10 years, 10 years to about 50 years, 25 years to about 100 years, or 50 years to about 130 years.

Also disclosed herein are kits comprising the pharmaceutical composition contained at least in part in packaging. Also disclosed herein are methods of making kits comprising a pharmaceutical composition contained at least in part in packaging.

Also disclosed herein are methods of treating a disease comprising treating the disease or condition by administering a therapeutically effective amount of the powdery pharmaceutical composition. Also disclosed herein are methods of treating a disease comprising treating the disease or condition by administering, via inhalation, a therapeutically effective amount of the powdery pharmaceutical composition. In some aspects, the disease may comprise treating a disease or condition selected from the group consisting of: anaphylaxis, asthma, a cardiac disorder, an allergic reaction, cardiac arrest, superficial bleeding, an infection, hypertension (e.g., increased blood pressure), hypotension, septic shock, attention deficit hyperactivity disorder (ADHD), depression, a mood disorder, a psychiatric disorder or any combination thereof. In some cases, a psychiatric disorder may comprise autism spectrum disorder, a communication disorder, a bipolar disorder, an anxiety disorder, a phobia, a stress-related disorder, a dissociate disorder, a somatic symptom disorder, an eating disorder, a sleep disorder, a disruptive disorder, a depressive disorder, a substance related disorder, a neurocognitive disorder, a schizophrenia, an obsessive-compulsive disorder, a personality disorder, or any combination thereof. In some cases, an infection may comprise a bacterial infection, a viral infection, a parasitic infection or a fungal infection. In some cases, an allergic reaction may comprise an allergy. In some instances, an allergy may be a drug (e.g., medication) allergy, a food allergy, an insect allergy, a vaccine allergy, a latex allergy, a mold allergy, a pet allergy, a plant allergy, a pollen allergy or any combination thereof. In some cases, a powdery pharmaceutical formulation disclosed herein may be administered to treat a seizure, such as an epileptic seizure. In some cases, a powdery pharmaceutical formulation disclosed herein may be administered to treat croup, shock or both. In some cases, a powdery pharmaceutical formulation disclosed herein may be administered to increase exercise performance, athletic performance, or both. In some cases, a powdery pharmaceutical composition disclosed herein can be used to treat conditions in the nasal cavity or the sinus cavity, for example in allergies and/or allergic rhinitis.

In some cases, a pharmaceutical composition disclosed herein can be administered to treat a diabetes. For example, an encapsulated insulin can be administered to treat a diabetes. In some cases, a diabetes can be Type 1 diabetes, Types 2 diabetes, a Type 3 diabetes, a Type 3c diabetes, gestational diabetes, or a combination thereof. In some cases, diabetes can comprise prediabetes.

In some cases, a powdery pharmaceutical formulation disclosed herein may be administered to treat a coronavirus infection, an influenza virus infection, a rhinovirus infection, an adenovirus infection, a parainfluenza virus infection, or a respiratory syncytial virus infection.

A coronavirus infection may comprise an infection by a virus in the subfamily Coronavirinae. In some cases, a coronavirus infection may comprise: an Alphacoronavirus, a Betacoronavirus, a Gammacoronavirus, or a Deltacoronavirus. In some instances, a coronavirus infection may comprise a 229E coronavirus, NL63 coronavirus, OC43 coronavirus, HKU1 coronavirus, severe acute respiratory syndrome coronavirus (SARS-CoV), severe acute respiratory syndrome coronavirus 2 (SARS-CoV-2) middle east respiratory syndrome-related coronavirus (MERS-CoV), a mutated form of any of these, or any combination thereof. SARS-CoV-2 may be also known as COVID-19, COVID-2019, 2019 novel coronavirus, or 2019-nCoV.

An influenza virus infection may comprise an infection by a virus in the subfamily Orthomyxoviridae. In some cases, an influenza virus infection may comprise: an Alphainfluenzavirus, a Betainfluenzavirus, or a Gammainfluenzavirus. In some cases, an influenza virus infection may comprise an infection by H1N1, H1N2, H2N2, H3N1, H3N2, H3N8, H5N1, H5N2, H5N3, H5N8, H5N9, H7N1, H7N2, H7N3, H7N4, H7N7, H7N9, H9N2, or a H10N7 strain.

A parainfluenza virus may comprise an infection by a virus in the subfamily Paramyxoviridae. In some cases, a parainfluenza virus is Human parainfluenza virus type 1, 2, 3, or 4.

A rhinovirus infection may comprise an infection by a virus in the subfamily Picornaviridae. In some cases, a rhinovirus infection may comprise rhinovirus A with a serotype of 1, 2, 7-13, 15, 16, 18-25, 28-34, 36, 38-41, 43-47, 49-51, 53-68, 71, 73-78, 80-82, 85, 88-90, 94-96, 98, or 100-103. In some cases, a rhinovirus infection may comprise rhinovirus B with a serotype of 3-6, 14, 17, 26, 27, 35, 37, 42, 48, 52, 69, 70, 72, 79, 83, 84, 86, 91-93, 97, or 99. In some cases, a rhinovirus infection may comprise rhinovirus C with a serotype of 1-51.

An adenovirus infection may comprise an infection by a virus in the subfamily Adenoviridae. In some cases, an adenovirus may comprise an Adenovirus A species of serotype of 12, 18, or 31. In some cases, an adenovirus may comprise an Adenovirus B species of serotype 3, 7, 11, 14, 16, 21, 34, 35, 50, or 55. In some cases, an adenovirus may comprise an Adenovirus C species of serotype 1, 2, 5, 6, or 57. In some cases, an adenovirus may comprise an Adenovirus D species of serotype 8, 9, 10, 13, 15, 17, 19, 20, 22, 23, 24, 25, 26, 27, 28, 29, 30, 32, 33, 36, 37, 38, 39, 42, 43, 44, 45, 46, 47, 48, 49, 51, 53, 54, 56, 58, 59, 60, 62, 63, 64, 65, 67, 69, 70, 71, 72, 73, 74, or 75. In some cases, an adenovirus may comprise an Adenovirus E species of serotype 4. In some cases, an adenovirus may comprise an Adenovirus F species of serotype 40 or 41. In some cases, an adenovirus may comprise an Adenovirus G species of serotype 52.

A respiratory syncytial virus (RSV) may comprise an infection by a virus in the subfamily Orthopneumovirus. In some cases. The RSV may comprise a RSV A subtype of clade GA1, GA2, GA3, GA4, GA5, GA6, GA7, GB1, GB2, GB3, or GB4. In some cases, the RSV may comprise a RSV B subtype of clade BA.

In some aspects, prior to treating, a subject may have been diagnosed with the disease. In some aspects, the subject may be a human, a man, a woman, an individual over 18 years of age, an individual under 18 years of age, or any combination thereof.

In some aspects, a subject may be from about 1 day to about 10 months old, from about 9 months to about 24 months old, from about 1 year to about 8 years old, from about 5 years to about 25 years old, from about 20 years to about 50 years old, from about 40 years to about 80 years old, or from about 50 years to about 130 years old.

In some aspects, a method may further comprise diagnosing a subject as having the disease. In some aspects, a diagnosing may comprise employing an in vitro diagnostic. In some aspects, the in vitro diagnostic may be a companion diagnostic.

In some aspects, a diagnosis may comprise a physical examination, a radiological image, a blood test, an antibody test, or any combination thereof. In some aspects, a diagnosis may comprise a radiological image and the radiological image may comprise: a computed tomography (CT) image, an X-Ray image, a magnetic resonance image (MRI), an ultrasound image, or any combination thereof.

In some aspects, a method may further comprise administering a second therapy to the subject. In some aspects, a second therapy may comprise acetaminophen, an opioid, a nonsteroidal anti-inflammatory drug, methotrexate, hydroxychloroquine, prednisone, cortisone, a biological response modifier, a cardiac drug, an antimicrobial drug, a salt of any of these, or any combination thereof. In some aspects, a second therapy may comprise a biological response modifier and the biological response modifier may comprise: abatacept, adalimumab, adalimumab-atto, anakinra, certolizumab pegol, etanercept, etanercept-szzs, golimumab, infliximab, infliximab-dyyb, rituximab, sarilumab, tocilizumab, a biologically active fragment of any of these, a salt of any of these, or any combination thereof. In some aspects, the second therapy may comprise a nonsteroidal anti-inflammatory drug and the nonsteroidal anti-inflammatory drug may comprise naproxen, ibuprofen, a salt of any of these, or any combination thereof. In some aspects, a composition may comprise an excipient, a diluent, a carrier, or any combination thereof.

In some aspects, the composition may be administered as needed, or for: one day, two days, three days, four days, five days, six days, a week, two weeks, three weeks, a month, two months, three months, four months, five months, six months, seven months, eight months, nine months, ten months, eleven months, a year, or chronically.

In some aspects, the composition may be administered so that the active ingredient or the pharmaceutically acceptable salt thereof in the unit dose ranges from about: 5 μg (micrograms) to about 1000 mg, 5 μg to about 50 μg, 40 μg to about 90 μg, 80 μg to about 120 g, 100 μg to about 150 μg, 140 μg to about 190 μg, 150 μg to about 220 μg, 200 μg to about 250 μg, 240 μg to about 300 μg, 290 μg to about 350 μg, 340 μg to about 410 μg, 400 μg to about 450 μg, 440 μg to about 500 μg, 500 μg to about 700 μg, 600 μg to about 900 μg, 800 μg to about 1 mg, 1 mg to about 5 mg, 1 mg to about 10 mg, 5 mg to about 15 mg, 12 mg to about 25 mg, 20 mg to about 50 mg, 40 mg to about 80 mg, 70 mg to about 100 mg, 90 mg to about 150 mg, 125 mg to about 250 mg, 200 mg to about 500 mg, 400 mg to about 750 mg, 700 mg to about 900 mg, or from about 850 mg to about 1000 mg. In some cases, the unit dose range may be more than about: 5 μg, 10 μg, 25 μg, 50 μg, 75 μg, 100 μg, 150 μg, 200 μg, 220 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, or 25 mg. In some cases, the unit dose range may be less than about: 5 μg, 10 μg, 25 μg, 50 μg, 75 μg, 100 μg, 150 μg, 200 μg, 220 μg, 250 μg, 300 μg, 350 μg, 400 μg, 450 μg, 500 kg, 550 kg, 600 kg, 650 kg, 700 kg, 750 kg, 800 kg, 850 kg, 900 kg, 950 kg, 1000 kg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, or 25 mg. In some cases, epinephrine or a salt thereof may be administered in a unit dose form of about 0.22 mg. In some cases, epinephrine or a salt thereof may be administered in a unit dose form of about 0.10, 0.20, 0.30, 0.40, or 0.50 mg. In some cases, an insulin may be administered in a unit dose form of about 20 μg. In some cases, an insulin may be administered in a unit dose form of about 10 μg, 20 μg, 30 μg, 40 μg, 50 μg, 60 μg, 70 μg, 80 μg, 90 μg, or 100 kg.

In some cases, an epinephrine or a salt thereof can be administered 1, 2, 3, 4, 5, or more times if necessary. In some cases, the time between an epinephrine dose can be about: 1 min, 5 min, 10 min, 20 min, 30 min, 40 min, 50 min, 1 hour or more.

In some cases, an insulin can be administered 1, 2, 3, 4, 5, or more times if necessary. In some cases, the time between an insulin dose can be about: 1 min, 5 min, 10 min, 20 min, 30 min, 40 min, 50 min, 1 hour or more.

Examples of powdery pharmaceutical compositions and methods of administration are shown in Table 1.

TABLE 1
Powdery pharmaceutical compositions
and methods of administration

| Active | | | |
|---|---|---|---|
| Pharmaceutical | Trade | Recommended | Route of |
| Ingredient | Name | Dosing Levels | Administration |
| Epinephrine | Epipen, | About 0.22 mg | Inhalation, |
| (adrenaline) | Adrenaclick, | | intranasal |
| | Andrenalin | | |
| Insulin | Insulin, | About 10-100 μg | Inhalation, |
| | Lantus, | (0.01-0.100 mg) or | intranasal |
| | Humulin, | about 20 μg | |
| | Humulog | | |
| | Novolin, | | |
| | Afrezza | | |

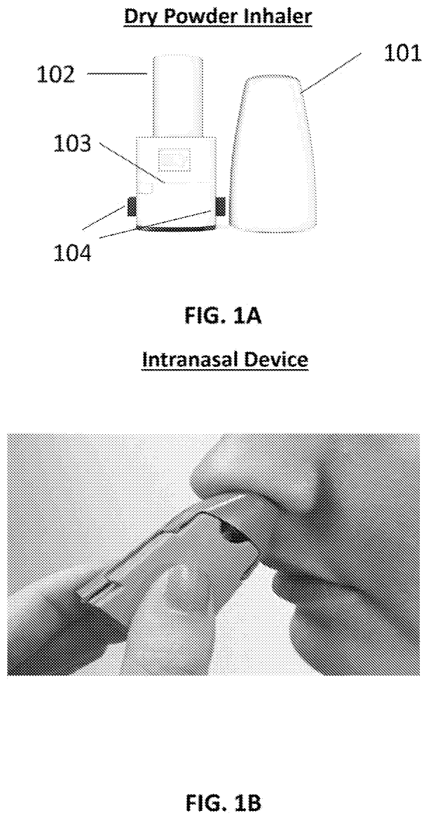

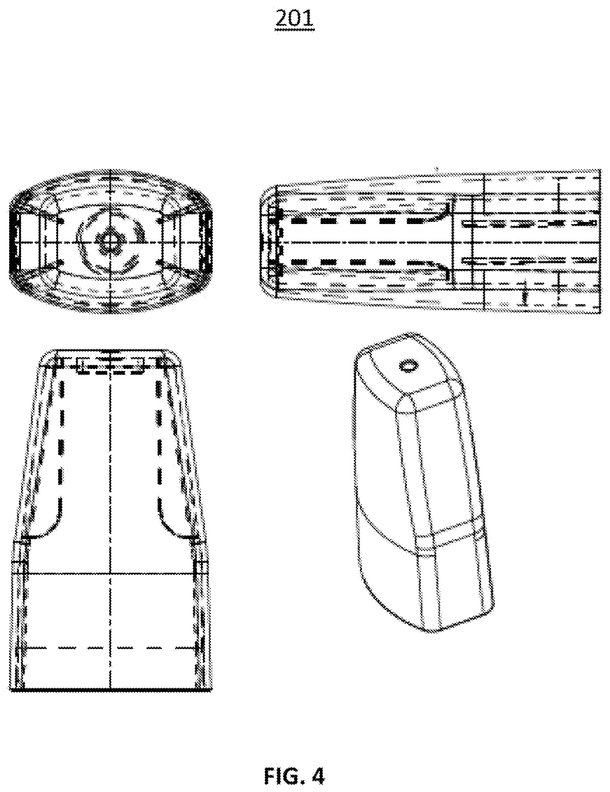

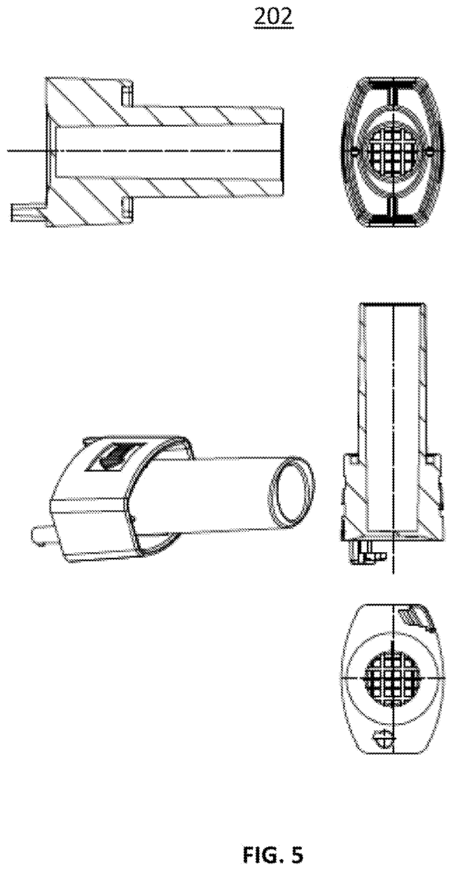

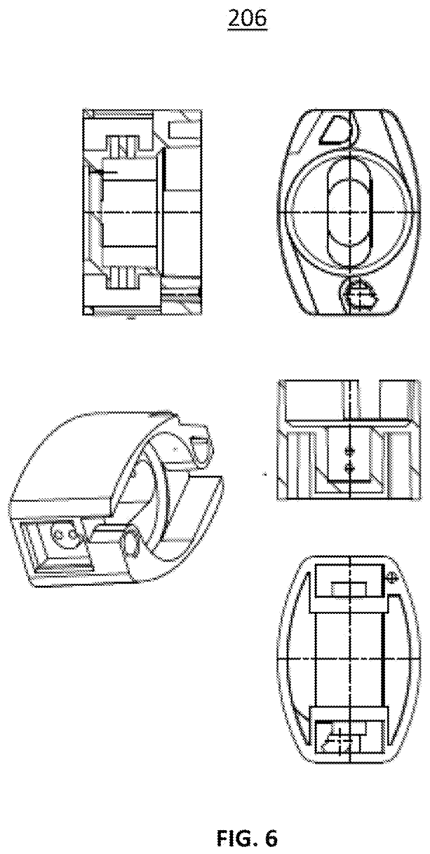

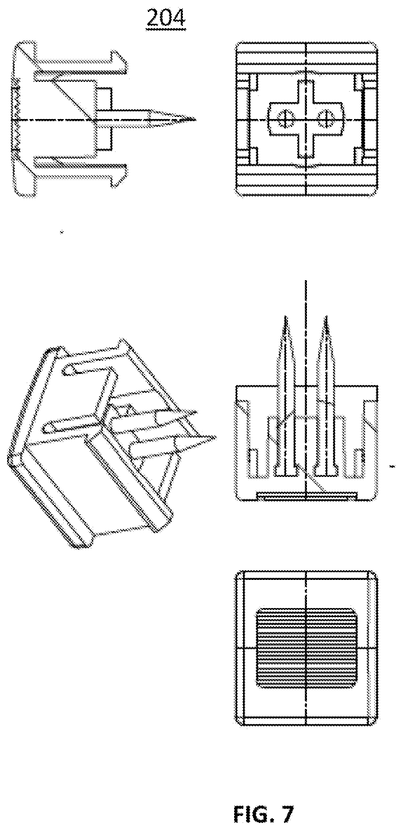

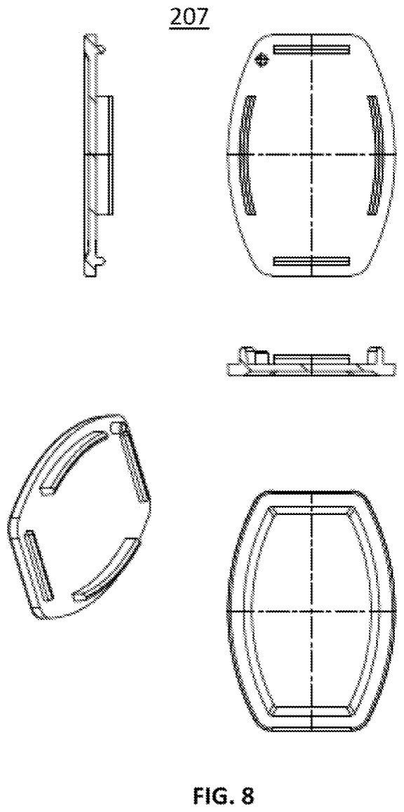

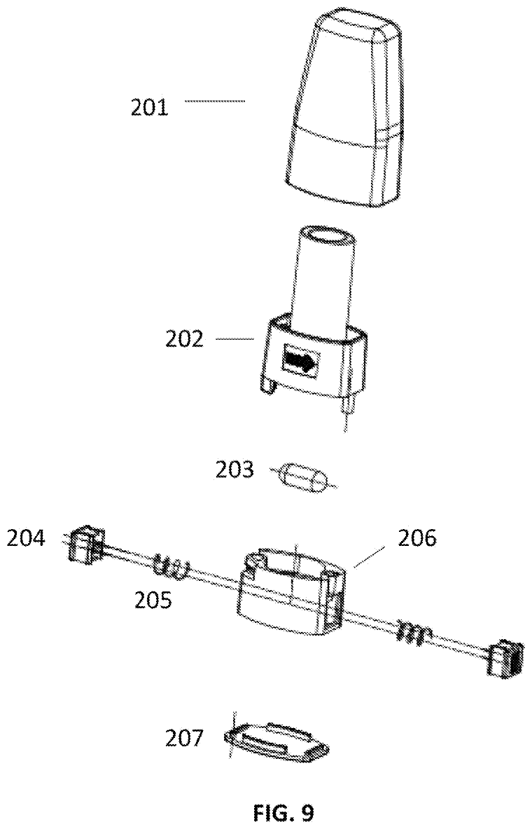

Referring to FIG. 1, FIG. 1A shows a dry powder inhaler device for delivery of powdery pharmaceutical compositions to the lung alveolar. The inhaler device may comprise a protective cap shown in FIG. 4, a rotatable top comprising a mouthpiece shown in FIG. 5, a lower base chamber receptacle for placing a pharmaceutical capsule shown in FIG. 6, a lateral button for mechanically piercing a capsule with a sharp surface while inside the chamber show in FIG. 7, and a chamber aerially connected to the mouthpiece permitting inhalation of capsule contents. The dry powder inhaler device may comprise a base plate as shown in FIG. 8. FIG. 9 shows a dry powder inhaler device with a protective cap, a rotatable comprising a mouthpiece, a lower base chamber for piercing a pill and a base plate.

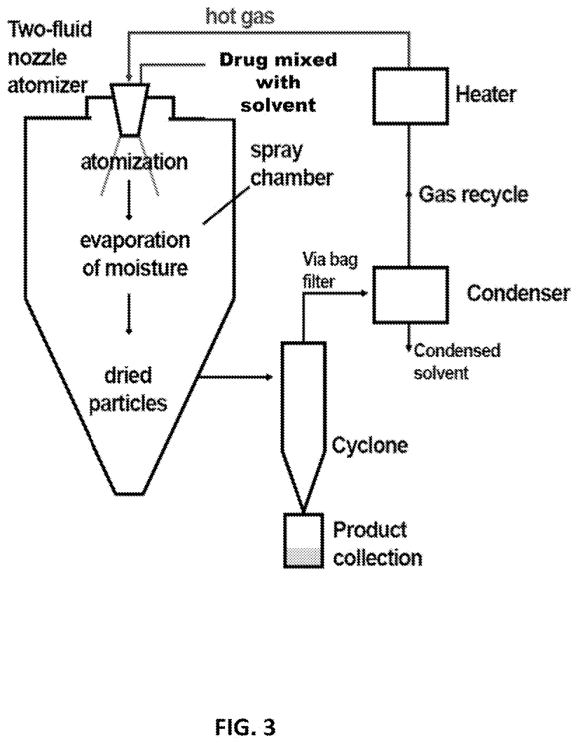

Referring to FIG. 3, FIG. 3 shows a spray drying manufacturing system comprising a closed spray dryer container which receives the solution comprising a drug dissolved or mixed in a suitable solvent (aqueous or solvent based). The solution then enters the particle formation chamber which is connected to an atomizer located at the top of the chamber. The atomizer is a two component or rotary nozzle type that distributes the solution into fine droplets controlled by the atomizer pressure. This atomization gas is an inert gas, either air or nitrogen. The atomized droplets go through a hot gas drying chamber to produce uniform fine particles that maintain a tight particle size distribution following liquid evaporation. Solid particle forms and falls to the bottom of the drying chamber. The balance between temperature, flow rate, and droplet size, controls the drying process. The powder is recovered from the exhaust gas using a cyclone or a bag filter. Particle size is validated by a Malvern particle analyzer prior to blending with an excipient carrier. The active powder is blended with an excipient carrier (lactose) product in a Patterson Kelly (PK Blender) and the blended powder is fed to a hopper. From the hopper, the dry powder is placed into a Size 3 Hypromellose capsule, by a Bosch Encapsulator machine.

In some aspects, a method of spray drying a liquid is disclosed herein. In some cases, a liquid may comprise i) epinephrine, or a pharmaceutically acceptable salt thereof or an insulin; ii) a coating material, wherein the coating material may comprise a hydroxypropyl methylcellulose (HPMC), a hydroxypropyl methylcellulose acetate succinate (HPMCAS), a cyclodextrin, a maltodextrin, a povidone, a copovidone or any combination thereof, and iii) a solvent. In some cases, the particles of the epinephrine or the pharmaceutically acceptable salt thereof may be dispersed in the liquid. In some cases, the particles of the insulin may be dispersed in the liquid. In some cases, the particles of the epinephrine or the pharmaceutically acceptable salt thereof dispersed in the liquid may have a particle diameter ranging from about 1 micrometer to about 5 micrometers once encapsulated. In some cases, the particles of the insulin dispersed in the liquid may have a particle diameter ranging from about 1 micrometer to about 5 micrometers once encapsulated. In some instances, the spray drying may comprise i) atomizing liquid droplets comprising the epinephrine or the pharmaceutically acceptable salt thereof, the coating material, and the solvent, ii) drying the droplets to form substantially encapsulated particles wherein the substantially encapsulated particles comprise the epinephrine or the pharmaceutically acceptable salt thereof substantially encapsulated by the coating material and iii) recovering the substantially encapsulated particles. In some instances, the spray drying may comprise i) atomizing liquid droplets comprising the insulin, the coating material, and the solvent, ii) drying the droplets to form substantially encapsulated particles wherein the substantially encapsulated particles comprise the insulin encapsulated by the coating material and iii) recovering the substantially encapsulated particles.

### Specific Embodiments

A number of compositions, and methods are disclosed herein. Specific exemplary embodiments of these compositions and methods are disclosed below. The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed.

### Specific Embodiments Section 1

Embodiment 1. A method of spray drying a liquid comprising contacting the following in a container:
a) particles of epinephrine or a pharmaceutically acceptable salt thereof,
b) a coating material, wherein the coating material comprises a hydroxypropyl methylcellulose (HPMC), a hydroxypropyl methylcellulose acetate succinate (HPMCAS), a povidone, a copovidone or any combination thereof, and
c) a solvent, wherein the particles of the epinephrine or the pharmaceutically acceptable salt thereof are at least partially dispersed in the solvent; and spray drying the liquid.

Embodiment 2. The method of embodiment 1, wherein the particles of the epinephrine or the pharmaceutically acceptable salt thereof at least partially dispersed in the solvent have a particle diameter ranging from about 1 micrometer to about 5 micrometers.

Embodiment 3. The method of embodiment 1 or 2, wherein the spray drying comprises i) atomizing liquid droplets comprising the epinephrine or the pharmaceutically acceptable salt thereof, the coating material, and the solvent, ii) drying the droplets to form substantially encapsulated particles, wherein the substantially encapsulated particles comprise the epinephrine or the pharmaceutically acceptable salt thereof substantially encapsulated by the coating material and iii) recovering the substantially encapsulated particles.

Embodiment 4: The method of embodiment 3, wherein the recovered particles of the epinephrine or the pharmaceutically acceptable salt thereof substantially encapsulated in the coating material have a particle diameter ranging from about 1 micrometer to about 10 micrometers, or from about 1 micrometer to about 5 micrometers, as measured by a particle analyzer using laser diffraction.

Embodiment 5. A powdery pharmaceutical composition, comprising: i) particles of a pharmaceutically acceptable excipient; and ii) particles comprising epinephrine or a pharmaceutically acceptable salt thereof, substantially encapsulated in a coating material, wherein the coating material comprises a hydroxypropyl methylcellulose (HPMC), a hydroxypropyl methylcellulose acetate succinate (HPMCAS), a povidone, a copovidone or any combination thereof, produced by a process comprising: a) mixing the particles comprising the epinephrine or the pharmaceutically acceptable salt thereof, the coating material, and a solvent; b) spray drying the mixed particles comprising epinephrine or the pharmaceutically acceptable salt thereof, the coating material, and the solvent to form the particles of ii) and blending the particles of i) and ii).

Embodiment 6. The powdery pharmaceutical composition of embodiment 5, wherein the spray drying comprises: a) atomizing liquid droplets comprising the epinephrine or the pharmaceutically acceptable salt thereof, the coating material, and the solvent, b) drying the droplets to form substantially encapsulated particles, wherein the substantially encapsulated particles comprise the epinephrine or the pharmaceutically acceptable salt thereof substantially encapsulated by the coating material and c) recovering the substantially encapsulated particles.

Embodiment 7. A method of making a powdery pharmaceutical composition, comprising contacting: i) particles of a pharmaceutically acceptable excipient; and ii) a plurality of spray dried particles, each particle of the plurality of spray dried particles comprising epinephrine or a pharmaceutically acceptable salt thereof, substantially encapsulated in a coating material, wherein a) at least a portion of the plurality of spray dried particles comprising the epinephrine or the pharmaceutically acceptable salt thereof substantially encapsulated in the coating material have a particle diameter ranging from about 1 micrometer to about 10 micrometers, or from about 1 micrometer to about 5 micrometers, as measured by a particle analyzer using laser diffraction and, b) wherein the coating material comprises a hydroxypropyl methylcellulose (HPMC), a hydroxypropyl methylcellulose acetate succinate (HPMCAS), a povidone, a copovidone or any combination thereof.

Embodiment 8. A method of making a powdery pharmaceutical composition, comprising contacting: i) particles of a pharmaceutically acceptable excipient; and ii) a plurality of spray dried particles, each particle of the plurality of spray dried particles comprising an insulin, substantially encapsulated in a coating material, wherein a) at least a portion of the plurality of spray dried particles comprising the insulin substantially encapsulated in the coating material have a particle diameter ranging from about 1 micrometer to about 10 micrometers, or from about 1 micrometer to about 5 micrometers, as measured by a particle analyzer using laser diffraction and, b) wherein the coating material comprises a hydroxypropyl methylcellulose (HPMC), a hydroxypropyl methylcellulose acetate succinate (HPMCAS), a povidone, a copovidone or any combination thereof.

### EXAMPLES

The following examples are included for illustrative purposes only and are not intended to limit the scope of the disclosure.

### Example 1

The powdery pharmaceutical composition described herein is administered by a dry powder inhaler or by a nasal inhaled device. Referring to FIG. 1A, FIG. 1A shows a dry powder inhaler device for delivery of a powdery pharmaceutical composition described herein to the lung alveolar. The inhaler device comprises: a protective cap 101, a rotatable top comprising a mouthpiece 102, a lower base chamber receptacle for placing the pharmaceutical capsule 103, lateral buttons for mechanically piercing the capsule with a sharp surface while inside the chamber 104, wherein the chamber is aerially connected to the mouthpiece permitting inhalation of capsule contents. FIG. 1B shows the nasal administration by a nasal inhaled device of a powdery pharmaceutical composition in a human subject. The composition is inhaled via the nares after the capsule containing the composition is pierced within the nasal inhaled device.

### Example 2

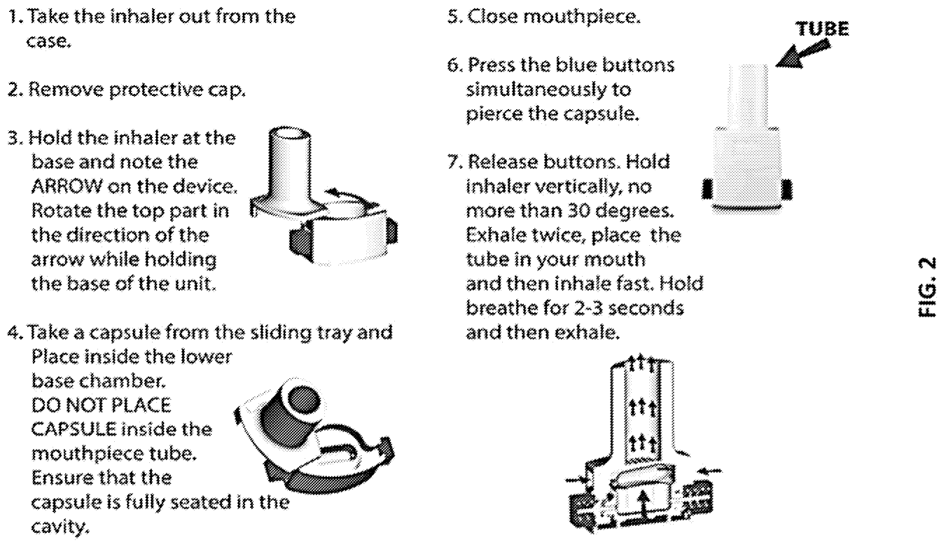

The method of using an inhaler device for the administration of a dry powdery pharmaceutical composition is shown in FIG. 2. The process for administration of the dry powdery pharmaceutical composition comprises 7 steps. Step 1: The inhaler is removed from the case. Step 2: The protective cap is removed. Step 3: The inhaler is held at the base and the top part is rotated in the direction of the arrow while the base of the unit is held. Step 4: A capsule is placed inside the lower base chamber cavity. Step 5: The mouthpiece is closed. Step 6: The buttons are pressed simultaneously to piece the capsule. Step 7: The buttons are released. The inhaler is held vertically, e.g., no more that about 30 degrees. The subject exhales twice before placing the tube in their mouth. The subject inhales quickly and holds their breath for about 2-3 seconds before exhaling.

### Example 3

The active ingredients (e.g., a sympathomimetic agent) in a dry powdery pharmaceutical composition described herein may be manufactured by a spray drying system. FIG. 3 shows a spray drying manufacturing system comprising a closed spray dryer container which receives the solution comprising a drug dissolved or mixed in a suitable solvent (aqueous and/or solvent based). The solution then enters the particle formation chamber which is connected to an atomizer located at the top of the chamber. The atomizer is a two component or rotary nozzle type that distributes the solution into fine droplets controlled by the atomizer pressure. This atomization gas is an inert gas or gas, such as air, nitrogen or carbon dioxide. The atomized droplets go through a hot gas drying chamber to produce uniform fine particles that maintain a tight particle size distribution following liquid evaporation. Solid particle form and fall to the bottom of the drying chamber. The balance between temperature, flow rate, and droplet size, controls the drying process. The powder is recovered from the exhaust gas using a cyclone or a bag filter. Particle size is validated by a Malvern particle analyzer prior to blending with an excipient carrier. The active powder is blended with an excipient carrier (lactose) product in a Patterson Kelly (PK Blender) and the blended powder is fed to a hopper. From the hopper, the dry powder is placed into a Size 3 Hypromellose capsule, by a Bosch Encapsulator machine.

### Example 4

A male subject will have an allergic reaction to shellfish. The subject is administered a pharmaceutical composition to treat the allergic reaction. The pharmaceutical composition comprises encapsulated epinephrine which is processed to a dry powder using the methods described herein (e.g., spay drying). The dry powder is mixed with a lactose powder and encapsulated. The epinephrine is packaged in a capsule and is administered intranasally with an inhaler. The dosing regimen comprises an effective amount (e.g., 0.22 mg) of epinephrine to treat the allergic reaction. The absorption of the inhaled pharmaceutical composition will reach the blood stream at least 5× faster than a comparable pharmaceutical composition that is administered orally.

### Example 5

A subject will be diagnosed with COVID-19. The subject is prescribed a dosing regimen of a pharmaceutical composition. The pharmaceutical composition comprises encapsulated epinephrine which has been processed to a dry powder using the methods described herein (e.g., spay drying). The dry powder is mixed with milled lactose and encapsulated. The pharmaceutical composition will be administered to the subject by inhalation administration. The dosing regimen comprises an effective amount (e.g., 0.22 mg) of epinephrine to treat the disease. A dosing level of inhaled epinephrine pharmaceutical composition will be about 90% lower than a subject receiving the oral administration of epinephrine.

### Example 6

The powdery pharmaceutical composition described herein is administered by a dry powder inhaler. Referring to FIG. 9, FIG. 9 shows a dry powder inhaler device for delivery of a powdery pharmaceutical composition described herein to the lung alveolar. The inhaler device comprises: a protective cap 201, a rotatable top comprising a mouthpiece 202, a lower base chamber receptacle 206 for placing the pharmaceutical capsule 203, lateral buttons for mechanically piercing the capsule with a sharp surface 204 while inside the chamber with the use of a spring 205, wherein the chamber is aerially connected to the mouthpiece permitting inhalation of capsule contents. The baseplate 207 is fitted to the lower base chamber receptacle.

### Example 7

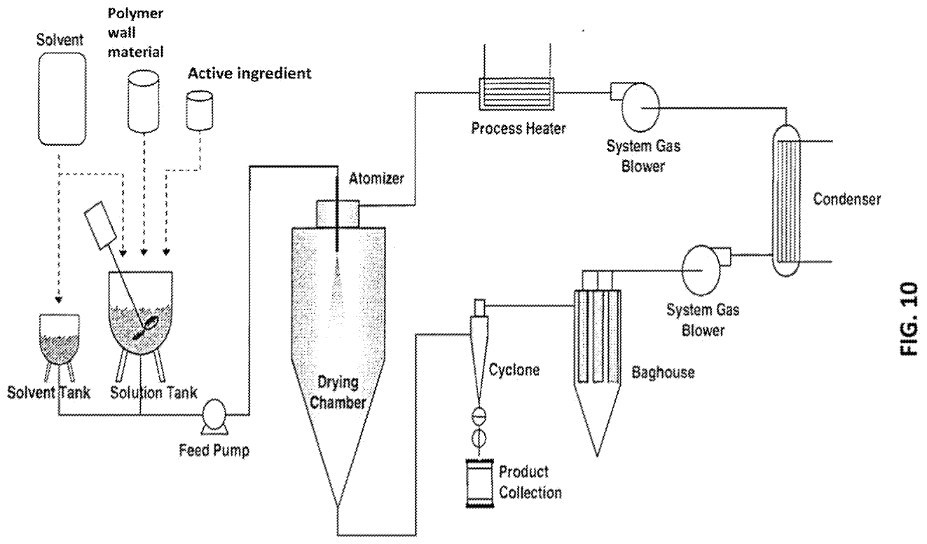

The active encapsulated ingredient epinephrine, in a dry powdery pharmaceutical composition described herein, was manufactured by a spray drying system. Epinephrine was encapsulated with HPMCAS, HPMC, trehalose, or mannitol using the methods described herein. In a particular example, FIG. 10 shows a spray drying manufacturing system comprising a closed spray drying chamber which received the solution comprising a polymer wall material (HPMCAS) dissolved in a solvent (e.g., 70% ethanol and 30% water) and an active ingredient (racepinephrine). The dissolved polymer wall material and the active ingredient were thoroughly mixed into a liquid suspension. The liquid suspension was fed into an atomizer located at the top of the chamber. The atomizer is a two component or rotary nozzle type that distributes the solution into fine droplets controlled by the atomizer pressure. The atomized droplets went through a drying chamber with hot gas to produce uniform fine particles that maintained a tight particle size distribution following liquid evaporation. Solid particles formed and fell to the bottom of the drying chamber as amorphous crystals. The balance between temperature, flow rate, and droplet size, controlled the drying process. The powder was recovered from the exhaust gas using a cyclone or a bag filter. The particle size of the encapsulated epinephrine particles was 1-5 microns. Particle size was validated by a Malvern particle analyzer prior to blending with an excipient carrier.

The active powder was blended with an excipient carrier (lactose) product in a blender and the blended powder was fed to a hopper. The excipient particle size was 75 to 100 μm. From the hopper, the dry powder was placed into a Size 3 Hypromellose capsule, by a Encapsulator machine.

### Example 8

Encapsulated insulin in a dry powdery pharmaceutical composition was manufactured by a spray drying as described herein. Insulin was encapsulated with HPMCAS, HPMC, trehalose, or HPC using the methods described herein. In a particular example, liquid human insulin was mixed with HPMCAS and a solvent (ethanol and water) and mixed at a low speed. The mixture was spray dried to create HMPCAS encapsulated human insulin.

### Example 9

A human subject is diagnosed with type 2 diabetes. The subject is administered a pharmaceutical composition to treat the diabetes. The pharmaceutical composition comprises encapsulated insulin which has been processed to a dry powder using the methods described herein (e.g., spay drying). The dry powder is mixed with a lactose powder and encapsulated. The insulin is packaged in a capsule and is administered by inhalation. The dosing regimen comprises an effective amount (e.g., 20 μg) of insulin to treat the diabetes.

### Example 10

A human subject was administered a composition comprising HPMCAS encapsulated epinephrine (particle size 1-5 μm) and a lactose excipient (75-100 μm) by inhalation with a dry powdered inhaler. The dose of the encapsulated epinephrine was 0.22 mg. The subject observed no side effects from the administration of the composition.

### Example 11

Human subjects were administered a composition comprising HPMCAS encapsulated insulin (particle size 1-5 μm) and a lactose excipient (75-100 μm) by inhalation with a dry powdered inhaler. The dose of the encapsulated insulin was: 15 μg, 50 μg, 75 μg or 100 μg. The subjects observed no side effects from the administration of the composition.

### Example 12

HPMCAS encapsulated insulin was tested for stability after spray drying. The physical and chemical properties of insulin remained intact and there was no degradation of the insulin during spray drying.

### Example 13

A human subject is diagnosed with an allergic reaction. The subject is administered a pharmaceutical composition to treat the allergic reaction. The pharmaceutical composition comprises encapsulated epinephrine which has been processed to a dry powder using the methods described herein (e.g., spay drying). The dry powder is mixed with a lactose powder and encapsulated. The epinephrine is packaged in a capsule and is administered by inhalation. The dosing regimen comprises an effective amount (e.g., 0.22 mg) of epinephrine to treat the allergic reaction.

### Example 14

HPMCAS encapsulated racepinephrine was tested for stability after spray drying. The physical and chemical properties of racepinephrine remained intact and there was no degradation of the racepinephrine during spray drying.

While preferred aspects of the present disclosure have been shown and described herein, it will be obvious to those skilled in the art that such aspects are provided by way of example only. Numerous variations, changes, and substitutions will now occur to those skilled in the art without departing from the disclosure. It should be understood that various alternatives to the aspects of the disclosure described herein may be employed in practicing the methods presented in the disclosure. It is intended that the following claims define the scope of the disclosure and that methods and structures within the scope of these claims and their equivalents be covered thereby.

What is claimed is:

1. A powdery pharmaceutical composition, comprising:
i) particles of a pharmaceutically acceptable excipient; and
ii) a plurality of spray dried particles, each particle of the plurality of spray dried particles comprising:
a) an epinephrine or a pharmaceutically acceptable salt thereof, or
b) an insulin or a derivative thereof,
wherein a) or b) is substantially encapsulated in a coating material,
wherein
within the plurality of spray dried particles at least a portion of the spray dried particles comprising the epinephrine or the pharmaceutically acceptable salt thereof, or the insulin or the derivative thereof, substantially encapsulated in the coating material, individually have a particle diameter ranging from about 1 micrometer to about 10 micrometers, as measured by a particle analyzer using laser diffraction and, wherein the coating material comprises a hydroxypropyl methylcellulose (HPMC), a hydroxypropyl methylcellulose acetate succinate (HPMCAS), a povidone, a copovidone or any combination thereof.

2. The powdery pharmaceutical composition of claim 1, comprising the plurality of spray dried particles, each particle of the plurality of spray dried particles comprising the epinephrine or a pharmaceutically acceptable salt thereof.

3. The powdery pharmaceutical composition of claim 1, comprising the plurality of spray dried particles, each particle of the plurality of spray dried particles comprising the insulin or the derivative thereof.

4. The powdery pharmaceutical composition of any one of claims 1-3, wherein the powdery pharmaceutical composition is for inhaled use or for intranasal use.

5. The powdery pharmaceutical composition of any one of claims 1-4, wherein the powdery pharmaceutical composition is in unit dose form.

6. The powdery pharmaceutical composition of any one of claims 1-5, wherein at least a portion of the particles of the pharmaceutically acceptable excipient individually have a particle diameter ranging from about 50 micrometers to about 200 micrometers, as measured by a particle analyzer using laser diffraction.

7. The powdery pharmaceutical composition of any one of claims 1-6, wherein the particles of i) and the plurality of spray dried particles of ii) are admixed into a substantially homologous mixture.

8. The powdery pharmaceutical composition of any one of claims 1-7, which is contained within a capsule.

9. The powdery pharmaceutical composition of claim 8, wherein the capsule is about one quarter to about one half, by volume, filled with the powdery pharmaceutical composition.

10. The powdery pharmaceutical composition of any one of claims 1-9, wherein a weight to weight ratio of: a) the particles of the pharmaceutically acceptable excipient and b) the particles comprising the epinephrine or the pharmaceutically acceptable salt thereof, substantially encapsulated in a coating material, ranges from about 1:1 (w/w) to about 10000:1 (w/w).

11. The powdery pharmaceutical composition of claim 10, wherein the weight to weight ratio of: i) particles of a pharmaceutically acceptable excipient and ii) the plurality of spray dried particles, ranges from about 1:1 (w/w) to about 10:1 (w/w).

12. The powdery pharmaceutical composition of any one of claims 8-11, wherein the portion of the capsule not containing the powdery pharmaceutical composition comprises a gas that at least partially comprises an inert gas.

13. The powdery pharmaceutical composition of claim 12, wherein the inert gas comprises nitrogen, carbon dioxide, helium, or any combination thereof.

14. The powdery pharmaceutical composition of claim 12 or 13, wherein the inert gas comprises at least about: 80%, 85%, 90%, or 95% of the gas on a volume to volume basis.

15. The powdery pharmaceutical composition of any one of claims 12-14, wherein the powdery pharmaceutical composition within the capsule, the gas within the capsule, or any combination thereof comprises less than about 10% water by weight based on the weight of the powdery pharmaceutical composition or a total content of all gases in the capsule is less than about 10% water by weight within: the powdery pharmaceutical composition within the capsule, the gas within the capsule, or any combination thereof.

16. The powdery pharmaceutical composition of any one of claims 8-15, wherein the capsule comprises a hydroxypropylmethylcellulose (HPMC) capsule.

17. The powdery pharmaceutical composition of any one of claims 8-16, wherein the capsule is size: 000, 00, 0, 1, 2, 3, or 4.

18. The powdery pharmaceutical composition of claim 17, comprising the capsule, wherein the capsule is size 3.

19. The powdery pharmaceutical composition of any one of claims 2-18, wherein in a human clinical trial, when inhaled into lungs, the powdery pharmaceutical composition operates mechanistically such that in at least a portion of the humans in the clinical trial, a majority of the particles of the pharmaceutically acceptable excipient deposit onto an oropharynx.

20. The powdery pharmaceutical composition of any one of claims 1-19, contained within an inhaler unit.

21. The powdery pharmaceutical composition of any one of claims 8-20, wherein the capsule is contained in an inhaler unit.

22. The powdery pharmaceutical composition of any one of claims 1-21, wherein the pharmaceutically acceptable excipient comprises a carbohydrate, an alginate, povidone, a carbomer, a flavor, a natural gum, a silicone, an alcohol, a butter, a wax, a fatty acid, a preservative, a pharmaceutically acceptable salt of any of these, or any combination thereof.

23. The powdery pharmaceutical composition of claim 22, wherein the pharmaceutically acceptable excipient or pharmaceutically acceptable salt thereof comprises the carbohydrate or the pharmaceutically acceptable salt thereof, and wherein the carbohydrate or the pharmaceutically acceptable salt thereof comprises lactose, microcrystalline cellulose, cellulose, mannitol, sorbitol, starch, starch glycolate, hydroxypropyl methylcellulose, hydroxypropyl methylcellulose acetate succinate, a cyclodextrin, maltodextrin, croscarmellose sodium, corn starch, carrageenan, sorbitol, maltitol, glucose, a pharmaceutically acceptable salt of any of these, or any combination thereof.

24. The powdery pharmaceutical composition of any one of claims 1-21, wherein the pharmaceutically acceptable excipient or the pharmaceutically acceptable salt thereof comprises a lactose or a pharmaceutically acceptable salt thereof.

25. The powdery pharmaceutical composition of claim 24, comprising the lactose or the pharmaceutically acceptable salt thereof, which comprises milled lactose, sieved lactose, micronized lactose, spray dried lactose, at least substantially anhydrous lactose, monohydrate lactose, a pharmaceutically acceptable salt thereof, or any combination thereof.

26. The powdery pharmaceutical composition of any one of claims 1-25, wherein the powdery pharmaceutical composition described herein when stored in a sealed container placed in a room at 25° C. and a room atmosphere having about 50 percent relative humidity, retains at least about: 90% of the epinephrine or the pharmaceutically acceptable salt thereof, or the insulin or the derivative thereof after 6 months, as measured by HPLC.

27. The powdery pharmaceutical composition of any one of claims 1, 2, or 4-26, wherein the epinephrine or the pharmaceutical acceptable salt thereof is present in an amount ranging from about 0.1 mg to about 0.5 mg.

28. The powdery pharmaceutical composition of any one of claims 1, 2, or 4-27, wherein the epinephrine or the pharmaceutically acceptable salt thereof is in the form of a pharmaceutically acceptable salt thereof and is a hydrochloride salt, a bitartrate salt or a borate salt.

29. The powdery pharmaceutical composition of any one of claims 1, 2, or 4-28, wherein the epinephrine or the pharmaceutically acceptable salt thereof comprises at least about 1% by weight of the overall powdery pharmaceutical composition.

30. The powdery pharmaceutical composition of any one of claims 1 or 3-26, wherein the insulin or the derivative thereof is present in an amount ranging from about 10 μg to about 100 μg.

31. The powdery pharmaceutical composition of any one of claims 1, 3-26 or 30, wherein the insulin or the derivative comprises a rapid-acting insulin, a short-acting insulin, an intermediate-acting insulin, a long-acting insulin, an ultra-long acting insulin, or a mixture thereof.

32. The powdery pharmaceutical composition of any one of claims 1-31, wherein the plurality of spray dried particles comprise a median diameter of about 1 μm to about 5 μm.

33. The powdery pharmaceutical composition of any one of claims 1-31, wherein the plurality of spray dried particles comprise a median diameter of about 1 μm to about:6 μm, 7 μm, 8 μm, or 9 μm.

34. A kit comprising the powdery pharmaceutical composition of any one of claims 1-33 contained at least in part in a packaging.

35. A method of treating a disease or condition in a subject in need thereof, comprising treating the disease or condition by administering, via inhalation, a therapeutically effective amount of the powdery pharmaceutical composition of any one of claims 1-33 to the subject in need thereof.

36. The method of claim 35, wherein the administering is conducted one, two, three, four, or more than four times per day.

37. The method of claim 35 or 36, wherein the disease or condition is selected from the group consisting of: anaphylaxis, asthma, a cardiac disorder, an allergic reaction, cardiac arrest, superficial bleeding, an infection, hypertension (e.g., increased blood pressure), hypotension, septic shock, attention deficit hyperactivity disorder (ADHD), depression, a mood disorder, a psychiatric disorder, a type I diabetes, a type II diabetes, a type III diabetes, a type IIIc diabetes, a gestational diabetes, and any combination thereof.

38. The method of any one of claims 35-37, wherein the powdery pharmaceutical composition is administered as needed, or for about: one day, two days, three days, four days, five days, six days, a week, two weeks, three weeks, a month, two months, three months, four months, five months, six months, seven months, eight months, nine months, ten months, eleven months, a year, or chronically.

39. The method of any one of claims 35-38, wherein a second therapeutic or pharmaceutically acceptable salt thereof is administered.

40. The method of claim 39, wherein the second therapeutic or a pharmaceutically acceptable salt thereof is administered concurrently or consecutively.

41. The method of claim 39, wherein the second therapeutic or the pharmaceutically acceptable salt thereof is comprised in the powdery pharmaceutical formulation.

42. The method of claim 39, wherein the second therapeutic or the pharmaceutically acceptable salt thereof is not comprised in the powdery pharmaceutical formulation.

43. The method of any one of claims 35-42, wherein the subject is diagnosed with the disease or condition.

44. The method of claim 43, wherein the diagnosing comprises employing an in vitro diagnostic.

45. The method of claim 44, wherein the in vitro diagnostic is a companion diagnostic.

46. The method of any one of claims 35-45, wherein the powdery pharmaceutical composition is contained within a capsule, wherein the capsule is at least in part contained within an inhaler, and wherein the inhaler contains a sharp surface configured to puncture or slice the capsule, and wherein, prior to administrating, the inhaler is actuated such that the sharp surface punctures or slices the capsule.

47. The method of any one of claims 35-46, wherein the inhalation is oral inhalation, intra nasal administration, or any combination thereof.

48. The method of any one of claims 35-47, wherein in a human clinical trial, the powdery pharmaceutical composition, when inhaled into lungs, provides in at least part of the humans in the clinical trial a time to peak plasma concentration (Tmax) of the epinephrine or the pharmaceutically acceptable salt thereof ranging from about 1 minute to about 10 minutes.

49. A method of increasing exercise performance in a subject in need thereof, comprising administering, via inhalation, a therapeutically effective amount of the powdery pharmaceutical composition of any one of claims 1, 2, 4-29, or 32-33 to the subject in need thereof.

50. A method of treating a diabetes in a subject in need thereof, comprising administering, via inhalation, a therapeutically effective amount of the powdery pharmaceutical composition of any one of claims 1, 3-26 or 30-33 to the subject in need thereof.
